(19) United States
(12) Patent Application Publication
(10) Pub. No.: US 2012/0197059 A1
(43) Pub. Date: Aug. 2, 2012

(54) UBIQUITIN INTERACTING MOTIF PEPTIDES AS CANCER THERAPEUTICS

(76) Inventors: YUNZHOU DONG, Edmond, OK (US); Hong Chen, Edmond, OK (US)

(21) Appl. No.: 13/362,702

(22) Filed: Jan. 31, 2012

Related U.S. Application Data
(60) Provisional application No. 61/438,020, filed on Jan. 31, 2011.

(51) Int. Cl. A61K 38/17; A61N 5/00; A61P 35/00; A61P 9/00; A61K 38/20; A61K 39/395

(52) U.S. Cl. 600/1; 514/19.3; 514/19.4; 514/19.5; 424/85.2; 424/133.1; 514/19.8; 514/21.4; 514/21.3

(57) ABSTRACT

The present invention involves the use peptides comprising ubiquitin interacting motifs (UIMs) alone or in combination with other agents to treat diseases involving neovascularization, such as cancer.

This application claims benefit of priority to U.S. Provisional Application Ser. No. 61/438,020, filed Jan. 31, 2011, the entire contents of which are hereby incorporated by reference.

The invention was made with government support under grants R01HL-093242-01, P20 RR018758-06, NS36251, CA46128, DK45735, P01HL085607 R01HL65978-5, R01HL077357-1 and P01HL070295-6 from the National Institutes of Health. The government has certain rights in the invention.

### BACKGROUND OF THE INVENTION

1. Field of the Invention

The present invention relates generally to the fields of oncology and anti-angiogenic therapy. More particularly, it concerns the use of UIM-containing peptides alone or in combination with other agents to treat cancer.

2. Description of Related Art

Angiogenesis is of fundamental importance for embryogenesis, organ growth and repair as well as many pathological conditions, such as ischemic heart disease and cancer (Carmeliet and Jain, 2000; Rossant and Howard, 2002; Jain, 2003; Carmeliet, 2003; Risau, 1997). Vascular development and angiogenesis in mammals require signaling through the vascular endothelial growth factor (VEGF) pathway, which stimulates processes that are regulated by Notch (Carmeliet, 2003; Risau, 1997; Olsson et al., 2006; Weinstein and Lawson, 2002; Jakobsson et al., 2009; Thurston and Kitajewski, 2008).

Crosstalk between VEGF and Notch signaling ensures functional angiogenesis in both physiological and pathological settings (Hellstrom et al., 2007; Williams et al., 2006; Ridgway et al., 2006; Noguera-Troise et al., 2006; Phng and Gerhardt, 2009; Thurston et al., 2007). Epsins, including epsin 1 and 2 are a family of evolutionally conserved endocytic clathrin adaptor proteins mediating endocytosis of specific ubiquitinated surface proteins (Wendland et al., 1999; Rosenthal et al., 1999; Chen et al., 1998; Wendland, 2002; Shih et al., 2002; Chen and De Camilli, 2005). Epsin 1 and 2 are expressed in all tissues with overlapping functions (Rosenthal et al., 1999; Chen et al., 1998; Chen et al., 2009). This redundancy is exemplified by normal life span of epsin 1 or 2 single knockout mice (KO) but embryonic lethality of epsin 1 and 2 double KO mice (DKO) (Chen et al., 2009).

Epsins contain characteristics common for general clathrin adaptor proteins; however, they are not essential for housekeeping forms of clathrin-mediated endocytosis, including transferrin and EGF receptors endocytosis, indicating a selective role in the endocytosis of specific cell surface cargos (Chen et al., 1998; Chen et al., 2009; Ford et al., 2002; Itoh et al., 2001; Traub, 2003; Chen and Zhuang, 2008; Kazazic et al., 2009; Overstreet et al., 2004). These cargos are generally ubiquitinated and recruited by epsins via their ubiquitin-interacting motifs (UIM) (Shih et al., 2002; Chen and De Camilli, 2005; Chen and Zhuang, 2008; Hawryluk et al., 2006; Hofmann and Falquet, 2001; Aguilar, 2003; Polo et al., 2002). Epsin DKO mice were defective in Notch signaling (Chen et al., 2009; Overstreet et al., 2004; Tian et al., 2004; Wang and Struhl, 2004), a pathway that had first shown to require the endocytic function of epsin by genetic studies in Drosophila (Overstreet et al., 2004; Tian et al., 2004; Wang and Struhl, 2004; Wang and Struhl, 2005). DKO embryos displayed multiorgan defects, including abnormal vascular development and angiogenesis (Chen et al., 2009).

### SUMMARY OF THE INVENTION

Thus, in accordance with the present invention, there is provided a method of treating cancer in a subject comprising administering to said subject an ubiquitin interactive motif (UIM)-containing peptide. The administration may be intra-tumoral, regional to a tumor, or systemic. The systemic administration may be oral, intravenous, or intaarterial. The cancer may be recurrent, metastatic or multidrug resistant. The cancer may be brain cancer, head & neck cancer, throat cancer, nasopharyngeal cancer, esophageal cancer, lung cancer, stomach cancer, liver cancer, pancreatic cancer, colon cancer, rectal cancer, prostate cancer, testicular cancer, ovarian cancer, uterine cancer, cervical cancer, breast cancer, or skin cancer.

Treating may comprise reducing tumor growth, reducing tumor size, reducing tumor burden, inducing apoptosis in cancer cells, inhibiting tumor tissue invasion, or inhibiting metastasis. The UIM-containing peptide may comprise the sequence X—Ac-Ac—Ac-Ac-Hy-X—X-Ala-X—X—X-Ser-X—X—Ac—X—X—X—X, where Hy represents a large hydrophobic residue (typically Leu), Ac represents an acidic residue (Glu, Asp), and X represents residues that are less well conserved. The method may further comprise a secondary anti-cancer therapy, such as radiation, surgery, chemotherapy, hormone therapy, immunotherapy, or toxin therapy. The secondary anti-cancer therapy may in particular be 2,4-disulfonyl phenyl tert-butyl nitrone (2,4-ds-PBN).

In another embodiment, there is provided a method of inducing non-productive vessel formation in a subject comprising administering to said subject an ubiquitin interactive motif (UIM)-containing peptide. The administration may be intra-tumoral, regional to a tumor, or systemic. The systemic administration may be oral, intravenous, or intaarterial.

The subject may have cancer, such as cancer that is recurrent, metastatic or multidrug resistant. The cancer may be brain cancer, head & neck cancer, throat cancer, nasopharyngeal cancer, esophageal cancer, lung cancer, stomach cancer, liver cancer, pancreatic cancer, colon cancer, rectal cancer, prostate cancer, testicular cancer, ovarian cancer, uterine cancer, cervical cancer, breast cancer, or skin cancer. The method may further include a second anti-cancer therapy.

The subject may have a non-cancer neovascular disease, such as retinal neovascular disease, such as wet macular degeneration, haemorrhagic telangiectasia (HHT), neurofibromatosis type 1, familial cavernous malformation, and forms of lymphangiogenesis. The method may comprises a secondary treatment for the non-cancer vascular disease, such as ruboxistaurine, VEGI IL-20, ranibizumab, bevacizumab or pegaptanib.

In still another embodiment, there is provided a pharmaceutical composition comprising a ubiquitin interactive motif (UIM)-containing peptide dispersed in a pharmalogically acceptable medium, carrier or diluent. The peptide may comprise the sequence X—Ac-Ac—Ac-Ac-Hy-X—X-Ala-X—X—X-Ser-X—X—Ac—X—X—X—X, where Hy represents a large hydrophobic residue (typically Leu), Ac represents an acidic residue (Glu, Asp), and X represents residues that are less well conserved. The peptide may be about 20-30 residues in length. The peptide may be 21, 22, 23, 24, 25, 26, 27, 28, 29 or 30 residues in length. The peptide may be formulated in a lipid carrier.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a device or a method that “comprises,” “has,” “contains,” or “includes” one or more elements possesses those one or more elements, but is not limited to possessing only those one or more elements or steps. Likewise, an element of a device or method that “comprises,” “has,” “contains,” or “includes” one or more features possesses those one or more features, but is not limited to possessing only those one or more features.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-N. Endothelial epsins are required for physiological and pathological angiogenesis. FIG. 1A, Whole mount E10 WT or EC-DKO embryos. FIG. 1B, Vascular abnormalities in the telencephalic region of EC-DKOs were revealed by whole-mount CD31 immunostaining of E10 WT or EC-DKO embryos. Arrows indicate regions of disorganized vasculature in EC-DKO embryos. FIGS. 1C, 1E, Whole-mount CD31 immunostaining of hindbrains of E9.5 WT or EC-DKO embryos (FIG. 1C) or the skin of P6 WT or EC-iDKO mice (FIG. 1E). CD31-positive surface areas in FIG. 1C and FIG. 1E were quantified by SlideBook software in FIG. 1D and FIG. 1F, respectively. FIG. 1G, Whole-mount isolectin B4 staining of retinal vessels of P6 WT or EC-iDKO mice. (FIG. 1H, Isolectin B4-positive surface area in g was quantified by SlideBook software. FIG. 1I, 3D confocal images of LLC tumor vessels by CD31 immunostaining revealed increased vascularity and more disorganized vessels in EC-iDKO tumor compared to WT. FIG. 1J, CD31-positive surface area in i was quantified by SlideBook software. FIG. 1K, Representative WT and EC-iDKO mice bearing LLC tumors at 18 days post inoculation of tumor cells. Dotted lines indicate tumors. FIG. 1L. Smaller tumor and reduced tumor growth in ECiDKO relative to WT mice. Inserts are representative WT and EC-iDKO tumors harvested at 18 days post inoculation of tumor cells. FIG. 1M, Lack of FITC-lectin perfusion of tumor vessels in ECiDKO relative to WT mice revealed by CD31 co-immunostaining. Arrows indicate FITC-lectin perfused tumor vessels. FIG. 1N, FITC-lectin-positive surface area in m was quantified by SlideBook software. *P<0.001 in FIGS. 1D, 1F, 1H; *P<0.0003 in FIG. 1L; *P<0.005 in FIGS. 1J, 1N, calculated using two-tailed Student's t-test. Error bars indicate the mean±s.e.m. n=8 in FIGS. 1D, 1F, 1H, 1N=10 in FIG. 1J, 1N; n=12 in FIG. 1L. Scale bars: FIG. 1A, 500 μm; FIG. 1B, 225 μm; FIG. 1C, FIG. 1E, FIG. 1G, FIG. 1M, 100 μm; FIG. 1I, 50 μm.

FIGS. 2A-N. Endothelial epsins control Notch and VEGFR-2 signaling and endothelial cell proliferation. FIG. 2A, RT-PCR showing impaired Notch signaling in EC-DKO embryo. FIG. 2B, Western blot showing deficient Notch signaling but increased total and phosphorylated VEGFR-2 in ECDKO embryo. FIG. 2C, Mouse endothelial cells (MECs) isolated from WT or EC-iDKO mice (DKO) were stimulated with VEGF-A (50 ng/ml) and VEGF signaling was analyzed by western blotting with epsin 1, VEGFR-2, PLCγ, ERK and tubulin antibodies and with phospho-specific antibodies to VEGFR-2 (pY1054/1059 or pY1175), PLCγ and ERK. FIG. 2D, Quantification of activation of VEGFR-2, PLCγ and ERK was performed using NIH ImageJ software. FIG. 2E, VEGF but neither FGF nor PDGF increased proliferation of DKO MECs measured by BrdU labeling. This increase is abrogated by inhibitors to VEGFR-2. FIG. 2F, Increased proliferation of ECs observed by in vivo BrdU labeling (green) in intestinal blood vessels immunostained with CD31 (red) in EC-iDKO relative to WT. FIG. 2G, BrdU-positive cells in CD31-positive area was quantified based on at least 30 randomly selected visual fields. FIGS. 2H-I, VEGF but neither FGF nor PDGF increased migration and proliferation of DKO MECs. WT or DKO MECs were subjected to a monolayer “wound injury” assay in the absence or presence of VEGF-A (50 ng/ml) (FIGS. 2H-I), FGF (25 ng/ml) (FIG. 21), and PDGF (25 ng/ml) (FIG. 21). Quantification of wound distance was performed using NIH ImageJ software. FIGS. 2J-M, VEGF but not FGF signaling is increased in DKO MECs. HUVEC transfected with either control or epsins 1 and 2 siRNAs were stimulated with VEGF-A (50 ng/ml) (FIGS. 2J-K) or FGF (25 ng/ml) (FIGS. 2L-M) and analyzed by western blotting with antibodies indicated. Growth factor-induced activation of signaling pathway was analyzed by western blotting with phospho-specific antibodies to VEGFR-2 (pY1054/1059 or pY1175), PLCγ, Akt and ERK. FIGS. 2K, 2M, Quantification of activation of VEGFR-2, PLCγ, Akt and ERK was performed using NIH ImageJ software. FIG. 2N, Restoring Notch signaling in DKO MECs with NICD slightly suppresses elevated VEGF-induced VEGFR-2 phosphorylation. DKO MECs were transfected with an empty vector or NICD for 24 h followed by stimulation with VEGF-A (50 ng/ml) and analysis using western blotting with VEGFR-2, phospho-specific VEGFR-2, NICD, epsin 1 and tubulin antibodies. *P<0.001 in FIGS. 2D-E; *P<0.005 in FIG. 2G; *P<0.003 in i; *P<0.001 in FIGS. 2K, 2M; calculated using two-tailed Student's t-test. Error bars indicate the mean±s.e.m. n=8 in FIG. 2D; n=6 in FIG. 2E; n=7 in FIG. 2G; n=5 in FIGS. 2I, 2K, 2M. Scale bars: FIG. 2F, 20 μm.

FIGS. 3A-I. VEGF stimulation induces epsin and VEGFR-2 interaction and ubiquitin-UIM interaction is required for VEGFR-2 binding to epsin and internalization. FIG. 3A, BAEC cells stimulated with VEGF-A (50 ng/ml) were immunoprecipitated with epsin 1 antibodies and western blotted with VEGFR-2 antibodies. FIG. 3B, Quantification of co-immunoprecipitated VEGFR-2 and total VEGFR-2 was performed using NIH ImageJ software. FIG. 3C, Lysates from HEK 293T cells expressing VEGFR-2 and Flag-epsin 1 or empty vector were immunoprecipitated with Flag antibodies and western blotted with VEGFR-2 and phospho-VEGFR-2 antibodies. FIG. 3D, Lysates from HEK 293T cells expressing VEGFR-2 and Flag-epsin 1 or empty vector were first immunoprecipitated with Flag antibodies and western blotted with ubiquitin and epsin 1 antibodies. Immunoprecipitates were eluted and subjected for second immunoprecipitation with VEGFR-2 antibodies and western blotted with ubiquitin and VEGFR-2 antibodies, suggesting that epsin 1 coprecipitated ubiquitinated VEGFR-2. FIG. 3E, Lysates from HEK 293T cells expressing VEGFR-2 and either wild-type HA-epsin 1 or HA-epsin 1ΔUIM, or empty vector were immunoprecipitated with HA antibodies and western blotted with VEGFR-2 antibodies, indicating that UIM is required for the interaction of epsin 1 with VEGFR-2. FIG. 3F, Lysates from HEK 293T cells expressing WT or a ubiquitin-deficient mutant of VEGFR-2 were immunoprecipitated with epsin 1 antibodies and western blotted with ubiquitin and VEGFR-2 antibodies, indicating that reduced ubiquitination abolishes the binding of the mutant VEGFR-2 to epsin 1. FIG. 3G, HEK 293T cells expressing WT or a ubiquitin-deficient mutant of VEGFR-2 were incubated with 100 ng/ml of biotinylated VEGF-A/Streptavidin Alexa Fluor 488 at 4° C. for 30 min, shifted to 37° C. for 0 to 15 min and processed for immunofluorescence. The ubiquitin-deficient mutant of VEGFR-2 failed to internalize upon VEGF stimulation. FIG. 3H, Wild-type but not a UIM-deficient mutant of epsin 1 suppressed elevated VEGF signaling in DKO MECs. DKO MECs were transfected with an empty vector, wild type epsin 1, or the UIM-deficient mutant of epsin 1 for 24 h followed by stimulation with VEGF-A (50 ng/ml) and analysis by western blotting with phospho-specific VEGFR-2, epsin 1 and tubulin antibodies. FIG. 3I, Quantification of phosphorylated VEGFR-2 was performed using NIH ImageJ software. *P<0.001 in FIG. 3B; *P<0.002 in FIG. 3I; calculated using two-tailed Student's t-test. Error bars indicate the mean±s.e.m. n=8 in FIG. 3D; n=10 in FIG. 3I. Scale bar: FIG. 3G, 10 μm. In FIGS. 3C-F, cells were stimulated with VEGF-A (50 ng/ml) for 2 min.

FIGS. 4A-T. Endothelial epsins are required for VEGF-induced VEGFR-2 internalization and degradation. FIGS. 4A, 4C, 4D, HUVEC were incubated with 50 ng/ml of VEGF-A for 0 to 30 min and processed for immunofluorescence. Colocalization of VEGFR-2 with epsin 1 at 2 min, EEA1 at 10 min, and CD63 at 20 min seen by confocal microscopy (FIG. 4A). Boxed region in a magnified in c. Quantification of colocalization in FIG. 4D. FIGS. 4B, 4E, 4F, HUVEC were incubated with 100 ng/ml of biotinylated VEGF-A/Streptavidin Alexa Fluor 488 at 4° C. for 30 min, shifted to 37° C. for 0 to 30 min and processed for immunofluorescence. Colocalization of biotinylated VEGF-A/Streptavidin Alexa Fluor 488-labeled VEGFR-2 with epsin 1 at 2 min, EEA1 at 10 min, and CD63 at 20 min seen by confocal microscopy (FIG. 4B). Boxed region in FIG. 4B magnified in FIG. 4E. Quantification of colocalization in FIG. 4F. FIGS. 4G-J, WT (FIG. 4G, 4I, 4J) or DKO MEC (FIG. 4H) were incubated with biotinylated VEGFA/Streptavidin Alexa Fluor 488 as in FIG. 4B. Colocalization of biotinylated VEGF-A/Streptavidin Alexa Fluor 488-labeled VEGFR-2 with Alexa Fluor 594-labeled epsin 1 at 2 min, EEA1 at 10 min, and LAMP1 at 20 min seen by confocal microscopy (FIGS. 4G-H). Boxed region in FIG. 4G magnified in FIG. 4I. Quantification of colocalization in FIG. 4J. Arrows indicate colocalization of the two proteins. *P<0.005 in FIG. 4D; *P<0.002 in FIG. 4F; *P<0.001 in FIG. 4J. Values are mean±s.e.m, obtained from five independent experiments performed in triplicate. FIGS. 4K-M, WT or DKO MECs were incubated with VEGF-A (50 ng/ml). Cell surface expression of VEGFR-2 was measured by ELISA assay (see Methods) (FIG. 4K) and internalized VEGFR-2 was determined by cleavable biotin labeling method (see Methods) (FIGS. 4L-M). FIG. 4M, Quantification of internalized VEGFR-2 in 1 was performed using NIH ImageJ software. FIG. 4N, MECs were incubated with 50 ng/ml of VEGF-A and processed for immunofluorescence using VEGFR-2, phospho-VEGFR-2, EEA1 and LAMP1 antibodies. Colocalization of phospho-VEGFR-2 with VEGFR-2 at 0.5 and 1 min, EEA1 at 5 and 10 min, and LAMP1 at 20 min seen by confocal microscopy. FIG. 4O, HUVEC transfected with either control or clathrin siRNAs were stimulated with VEGF-A (50 ng/ml) and analyzed by western blotting with clathrin heavy chain, VEGFR-2, phospho-VEGFR-2, and tubulin antibodies. FIG. 4P, Quantification of activation of VEGFR-2 was performed using NIH ImageJ software. FIG. 4Q, HUVEC transfected with either control or dynamin 2 siRNAs were stimulated with VEGF-A (50 ng/ml) for the time points indicated. Cell lysates were analyzed by western blotting with dynamin 2, VEGFR-2, phospho-VEGFR-2, PLCγ, phospho-PLCγ, Akt, phospho-Akt, ERK, phospho-ERK and tubulin antibodies. FIG. 4R, Quantification of activation of VEGFR-2, PLCγ, Akt and ERK was performed using NIH ImageJ software. FIGS. 4S-T, HUVEC transfected with either DMSO, 40 μM Dynasore (FIG. 4S) or 80 μM Dynasore (FIG. 4T) were stimulated with VEGF-A (50 ng/ml) for the time points indicated. Cell lysates were analyzed by western blotting with VEGFR-2, phospho-VEGFR-2, phospho-PLCã, phospho-Akt, phospho-ERK and tubulin antibodies. *P<0.005 in FIG. 4K; *P<0.001 in FIG. 4M; *P<0.003 in FIG. 4P; *P<0.004 in FIG. 4R; calculated using two-tailed Student's t-test. Error bars indicate the mean±s.e.m. n=8 in FIG. 4K, 4M; n=5 in FIGS. 4P, 4R. Scale bars: 10 μm in FIGS. 4A, 4B, 4G, 4H, 4N.

FIGS. 5A-E. Generation of conditional Epn1flox/flox mice and EC-DKO or EC-iDKO mice. FIG. 5A, Diagram shows homologous recombination of the floxed gene-targeting vector at the Epn1 locus. Wild-type Epn1 allele, top row; targeting construct, second row; targeted Epn1 allele, third row; Epn1 floxed allele without Neo cassette (Epn1fl), bottom row. FIG. 5B, Strategy to generate constitutive endothelial cell-specific epsin double knockout mice (EC-DKO) by crossing Epn1fl/fl, Epn2−/− mice with Tie2 Cre deleter mice which specifically inactivate epsin 1 gene in endothelial and hematopoietic cells. FIG. 5C, Strategy to generate tamoxifen inducible endothelial cell-specific DKO mice (EC-iDKO) by crossing Epn1fl/fl, Epn2−/− mice with VEcad-ERT2 Cre deleter mice, which specifically inactivate epsin 1 gene in endothelial cells upon tamoxifen administration. FIG. 5D, Genomic PCR analysis of DNA isolated from mice tails. Genotypes for Epn1 of each mouse are indicated. FIG. 5E, Lysates from endothelial cells isolated from WT or EC-iDKO (DKO) mice were treated with tamoxifen followed by western blot analysis for epsin 1 and epsin 2 (not shown). Neither epsin 1 nor epsin 2 can be detected in DKO EC.

FIGS. 6A-G. Increased embryonic and postnatal angiogenesis by loss of endothelial epsins. FIG. 6A, Whole-mount CD31 immunostaining of midbrain region of E10 WT or EC-DKO embryos showing enhanced vascular network and increased number and diameter of blood vessels in EC-DKO embryos relative to WT. CD31-positive surface area in was quantified by SlideBook software in FIG. 6D. Error bars indicate the mean±s.e.m. n=5. FIG. 6B, CD31 immunostaining of cross sections of E10 WT or EC-DKO embryos hindbrains showing a more fully elaborated subventricular vascular plexus. CD31-positive surface area was quantified by SlideBook software in FIG. 6E. Error bars indicate the mean±s.e.m. n=8. FIG. 6C, Whole-mount Isolectin B4 staining of retinal vessels of WT or EC-iDKO mice at P6. Isolectin B4-positive surface area was quantified by SlideBook software in FIG. 6F. Error bars indicate the mean±s.e.m. n=10. Boxed enlarged images show increased sprouting in EC-iDKO relative to WT. The number of sprouts was quantified based on that at least 30 randomly selected visual fields were examined for each sample and at least 100 sprouts were counted for each visual field. FIG. 6G, Decreased LLC tumor incidence in EC-iDKO relative to WT mice. n=30. Error bars indicate the mean±s.e.m. Scale bars: FIGS. 6A-C, 50 μm; insert in FIG. 6C, 10 μm.

FIGS. 7A-F. Increased VEGF but neither FGF nor PDGF signaling both in vitro and in vivo by loss of endothelial epsin. FIG. 7A, Mouse endothelial cells isolated from wild type (WT) or ECiDKO mice (DKO) were stimulated with VEGF-A (50 ng/ml) for the time points indicated and cell membrane fractions were analyzed by western blotting with antibodies indicated. Note increased cell surface expression and enhanced phosphorylation of VEGFR-2 in DKO relative to WT MECs. FIG. 7B, HUVEC transfected with either control or epsins 1 and 2 siRNAs were stimulated with PDGF (25 ng/ml) for the time points indicated. Cell lysates were analyzed by western blotting with epsin 1, epsin 2, ERK, phospho-ERK and tubulin antibodies. FIG. 7C, Quantification of activation of ERK was performed using NIH ImageJ software. Error bars indicate the mean±s.e.m. n=5. FIG. 7D, WT or EC-iDKO P6 retina were immunostained with Isolectin B4 after IP/Intraocular injection of saline or anti-VEGFR-2, inhibitors to VEGFR-2 (not shown), FGFR and PDGFR, showing that elevated angiogenic sprouting in EC-iDKO can only be reversed by administration of anti-VEGFR-2 or inhibitors to VEGFR-2 but not to either FGFR or PDGFR. Administration of FGFR or PDGFR inhibitors blocked FGF (FIG. 7E) or PDGF (FIG. 7F)-induced signaling shown by western blotting analysis of phospho-Akt in skin and heart tissue samples. Scale bars: 50 μm.

FIGS. 8A-F. VEGF-induced endothelial cell migration and proliferation is enhanced due to loss of epsin. FIG. 8A, HUVEC transfected with either control or epsins 1 and 2 siRNAs were subjected to a monolayer “wound injury” assay in the absence or presence of VEGF-A (50 ng/ml) for 12 h. FIG. 8B, Quantification of wound distance in a at 12 h was performed using NIH ImageJ software. Error bars indicate the mean±s.e.m. n=3. FIG. 8C, HUVEC transfected with either control or epsins 1 and 2 siRNAs were cultured on Matrigel for 16 h in the absence or presence of VEGF-A (50 ng/ml). FIG. 8D, Quantification of tube formation (capillary-like networks) in FIG. 8C at 16 h was performed using NIH ImageJ software. Error bars indicate the mean±s.e.m. n=3. *P=0.02 in (FIG. 8B), *P=0.03 in FIG. 8D. FIG. 8E, WT or DKO MECs were cultured on Matrigel for 12 h in the absence or presence of VEGF-A (50 ng/ml). FIG. 8F, Quantification of network formation in (FIG. 8E) at time points indicated was performed using NIH ImageJ software. Error bars indicate the mean±s.e.m. n=6.

FIGS. 9A-D. Restoring Notch signaling does not significantly decrease enhanced VEGF-induced endothelial cell migration and proliferation in DKO MECs; block Notch signaling does not cause dramatic increase in VEGF signaling in WT MECs and angiogenesis in WT skin. FIG. 9A, Restoring Notch signaling in DKO MECs with NICD slightly suppresses elevated VEGF-induced VEGFR-2 phosphorylation and MEC migration and proliferation. DKO MECs were transfected with an empty vector or NICD for 24 h followed by subjecting to a “wound injury” assay to assess EC migration and proliferation. FIG. 9B, γ-secretase inhibitors treatment did not result in dramatic elevated VEGF-induced VEGFR-2 phosphorylation in WT MECs. WT MECs were treated with or without α-secretase inhibitors (10 μM) for 24 h followed by stimulation with VEGF-A (50 ng/ml) for the time points indicated. Cell lysates were analyzed by western blotting with VEGFR-2, phospho-specific VEGFR-2, NICD and tubulin antibodies. γ-secretase inhibitors treatment blocks NICD production but does not lead to the same dramatic increase in VEGF-induced VEGFR-2 phosphorylation in WT MECs as seen in DKO MECs, FIG. 9C, Normal angiogenesis in skin of WT mice treated with γ-secretase inhibitors. Wild-type pups were injected intraperitoneally with 100 mg/kg (body weight) of γ-secretase inhibitors per day from postnatal day 1 (P1) to P3. Pups were euthanized at P6 and skin from abdomen was harvested, processed for immunofluorescence staining with CD31 antibodies. FIG. 9D, Reduced generation of NICD after γ-secretase inhibitors administration was shown by western blot analysis of skin tissue samples. Scale bar: 100 μm.

FIGS. 10A-D. Epsin binds wild-type but not ubiquitin-deficient mutant VEGFR-2. FIG. 10A, Lysates from BAEC cells stimulated with VEGF-A (50 ng/ml) for the time points indicated were immunoprecipitated with VEGFR-2 antibodies or control IgG and western blotted with antibodies indicated. FIG. 10B, Lysates from HEK 293T cells expressing Flag-epsin 1 and VEGFR-2 or empty vector were immunoprecipitated with VEGFR-2 antibodies and western blotted with Flag antibodies, showing that VEGFR-2 coprecipitate epsin 1. FIG. 10C, Lysates from HEK 293T cells expressing wild-type VEGFR-2 or a ubiquitin-deficient mutant of VEGFR-2 and HA-epsin 1 or empty vector were immunoprecipitated with VEGFR-2 antibodies and western blotted with epsin 1 antibodies. FIG. 10D, Reduced binding of epsin 1 to the ubiquitin-deficient VEGFR-2 mutant. Error bars indicate the mean±s.e.m. n=5. *P<0.01.

FIGS. 11A-D. VEGFR-1 does not undergo VEGF-induced endocytosis and VEGFR-3 is not expressed in blood endothelial cells. FIG. 11A, HUVEC were incubated with 50 ng/ml of VEGF-A for 2 min and processed for immunofluorescence. Colocalization of VEGFR-2 with clathrin seen by confocal microscopy. FIG. 11B, WT or DKO MECs were incubated with VEGF-A (50 ng/ml) for 0, 10 and 20 m. Internalized VEGFR-1 was determined by cleavable biotin labeling method (see Methods). Note that no internalization of VEGFR-1 was observed in WT or DKO MECs. FIG. 11C, MECs were incubated with 100 ng/ml of biotinylated VEGF-A/Streptavidin Alexa Fluor 488 at 4° C. for 30 min, shifted to 37° C. for 0 to 10 min and processed for immunofluorescence using VEGFR-1 antibody. FIG. 11D, Lysates from HUVEC and MECs were subjected to western blotting analysis using antibodies against VEGFR-1, VEGFR-2, VEGFR-3 and tubulin. No expression of VEGFR-3 was detected in either HUVEC or MECs. Scale bar: 10 μm.

FIGS. 12A-C. Melanoma tumor model. (FIG. 12A) Dorsal view of tumor size. (FIG. 12B) Tumor volume over time. (FIG. 12C) Tumor volume following excision. n=3 in PBS group; n=4 in UIM group; p<0.05

FIG. 13. UIM treatment significantly increases tumor hypoxia and necrosis

FIGS. 14A-B. UIM treatment significantly delays melanoma tumor incidence. (FIG. 14A) Tumor incidence over time. (FIG. 14B) Percent tumor-free mice.

FIG. 15. UIM treatment inhibits LLC tumor growth. After 3 days of implantation, IV injection of UIM D-isomer peptide at 200 μg/mouse daily is started. Tumor initiation in UIM-treated mice delayed 2 days versus PBS; n=5.

FIGS. 16A-B. UIM treatment inhibits prostate tumor growth. IP injection of UIM peptide at 200 μg/mouse twice per week for five weeks; n=6-8.

FIG. 17. UIM peptide injection significantly inhibits tumor growth and improves prostate quality and surrounding vesicles.

FIG. 18. UIM increases survival rate of mice bearing brain tumors. After 7 days of implantation, UIM peptide is administered IV at 200 μg/mouse daily. Tumor growth is monitored every two or three days by MRI; n=6-8.

FIG. 19. Quantification of VEGFR2 signal intensity. UIM upregulates VEGFR2 level in brain tumor area revealed by functional and molecular-targeting MRI using anti-VEGFR2 probe.

FIG. 20. UIM treatment significantly increases VEGFR2 in GL6 brain tumors. Control=3; UIM=5; p<0.05

FIG. 21. UIM treatment increases survival rate of mice bearing brain tumors. Control=2; UIM=6.

### DESCRIPTION OF ILLUSTRATIVE EMBODIMENTS

As discussed below, the inventors now show that in addition to essential functions in Notch activation, epsins also have a key role in regulating VEGF signaling by promoting VEGFR-2 internalization and signaling switch off. VEGF signaling is critical in normal angiogenesis, including wound healing and tissue repair, but it also is important in pathological conditions, such as ischemia, diabetes and cancer. The studies described below provide new insight into the regulation of VEGF action in angiogenesis, and implicate targeting of epsin as a therapeutic strategy for a variety of diseases involving a vascular component. These and other aspects of the invention are described in detail below.

### 1. EPSINS AND UIM PEPTIDES

A. Epsins

Epsins are the family of membrane proteins that are important in creating the needed membrane curvature. Epsins contribute to various needed membrane deformations like endocytosis and block vesicle formation during mitosis. Epsins has many different domains to interact with various proteins related to endocytosis. At its N-terminus is an ENTH domain situated that binds Phosphatidylinositol (4,5)-bisphosphate what means it binds a lipid of biological membranes. Further this is a possible site for cargo-binding. In the middle of the epsin sequence are two UIM's (ubiquitin-interacting motifs) located. The C-terminus contains multiple binding sites, for example for clathrin and AP2 adaptors. Because of that Epsins are able to bind to a membrane with a specific cargo and connect it with the endocytosis machinery, so you may understand Epsin as something like a Swiss army knife for endocytosis. They may be the major membrane curvature driving proteins in many clathrin-coated vesicle budding events. Epsin 4 which encodes the protein Enthoprotin, now known as Clathrin Interactor 1 (CLINT1) has been shown to be involved in the genetic susceptibility to schizophrenia in four independent studies. A genetic abnormality in CLINT1 is assumed to change the way internalisation of neurotransmitter receptors occurs in the brains of people with schizophrenia.

B. UIMs

The recognition of ubiquitylated proteins is frequently mediated by conserved ubiquitin binding modules, which include the ubiquitin interacting motif (UIM). UIM permits binding of molecules containing such motifis to ubiquitin. UIM was originally identified based upon studies of the S5a subunit of the 19 S regulator in the human 26 S proteasome. Biochemical and mutational analyses revealed two copies of a ˜30-residue sequence motif (initially denoted pUbS) that can bind ubiquitylated protein and polyubiquitin chains. The pUbS motifs have hydrophobic core sequences composed of alternating large and small residues (Leu-Ala-Leu-Ala-Leu) that are flanked on both sides by patches of acidic residues. A more general definition of UIM, found in a number of different proteins that function in a variety of biological pathways, provides that UIM is a 20 residue sequence corresponding to the consensus: X—Ac-Ac—Ac-Ac-Hy-X—X-Ala-X—X—X-Ser-X—X—Ac—X—X—X—X, where Hy represents a large hydrophobic residue (typically Leu), Ac represents an acidic residue (Glu, Asp), and X represents residues that are less well conserved.

UIMs are particularly prevalent in proteins that function in the pathways of endocytosis and vacuolar protein sorting, which serve to sort membrane-associated proteins and their cargo from the plasma membrane (or Golgi) for eventual destruction (or localization) in the lysosome (yeast vacuole). Endocytic proteins that contain UIMs include the epsins, including Eps15 and Eps15R. These proteins are required for endocytosis of receptor: ligand complexes, including the complex of the epidermal growth factor (EGF) with its receptor (EGFR). UIMs can both bind ubiquitin and also direct protein ubiquitylation, although the relationship between these two activities is not yet fully understood.

C. UIM Peptides

A peptide is generally considered to be a small polypeptide having no more than about 30-40 residues, more typically no more than about 30 residues, such as 20-30 residues in length, including 20, 21, 22, 23, 24, 25, 26, 27, 28, 29, and 30, and ranges from 20 residues upward to each of the aforementioned individual numbers as upper limits. Also contemplated are truncated peptides comprising less than 20 residues that still retain ubiquitin binding activity, such as 6, 7, 8, 9, 10, 11, 12, 13, 14, 15, 16, 17, 18, 19 residue peptides.

As discussed above, a general definition for UIM is a 20 residue sequence corresponding to the consensus: X—Ac-Ac—Ac-Ac-Hy-X—X-Ala-X—X—X-Ser-X—X—Ac—X—X—X—X, where Hy represents a large hydrophobic residue (typically Leu), Ac represents an acidic residue (Glu, Asp), and X represents residues that are less well conserved. A more restrictive definition is a peptide containing alternating Leu and Ala residues (Leu-Ala-Leu-Ala-Leu).

D. Purification of Proteins

It may be desirable to purify UIM peptides, peptide-mimics or analogs thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

E. Peptide Synthesis

UIM-containing peptides may be generated synthetically for use in various embodiments of the present invention. Because of their relatively small size, the peptides of the invention can be synthesized in solution or on a solid support in accordance with conventional techniques. Various automatic synthesizers are commercially available and can be used in accordance with known protocols. See, for example, Stewart & Young, (1984); Tam et al., (1983); Merrifield, (1986); Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

### 2. THERAPY

The present invention envisions the use of the claimed UIM-containing peptides for the treatment of cancer and other diseases characterized by pathologic neovascularization. In particular, as explained herein, these peptides interfere with the normal interactions between epsins and VEGF and VEGFR-2, thereby disturbing the angiogenic processes driven by tumor formation. As a result, aberrant and non-functional vessels are produced that serve to impair blood flow to, e.g., a growing tumor and thus inhibit both its growth and spread.

Thus, in one aspect, the present invention seeks to treat cancers. The types of cancers are not limited except that they should have a vascular component, and thus would include any solid tumor such as brain cancer, head & neck cancer, throat cancer, nasopharyngeal cancer, esophageal cancer, lung cancer, stomach cancer, liver cancer, pancreatic cancer, colon cancer, rectal cancer, prostate cancer, testicular cancer, ovarian cancer, uterine cancer, cervical cancer, breast cancer, or skin cancer.

In addition to cancer, the present application also provides methods of treating non-cancer disease states that involve abnormal vascular development. In particular, abnormal vascular development is a contributing factor in certain diseases of the retina. Other disease of vascular malformation include hereditary haemorrhagic telangiectasia (HI-IT), neurofibromatosis type 1, familial cavernous malformation, and forms of lymphangiogenesis.

A. Formulations

The present invention discloses peptides numerous compositions, which in certain aspects of the invention, are administered to animals. For example, UIM peptides will be formulated for administration. Where clinical applications are contemplated, it will be necessary to prepare pharmaceutical compositions of these compounds and compositions in a form appropriate for the intended application. Generally, this will entail preparing compositions that are essentially free of pyrogens, as well as other impurities that could be harmful to humans or animals.

One will generally desire to employ appropriate salts and buffers to render agents suitable for introduction into a patient. Aqueous compositions of the present invention comprise an effective amount of the agent, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients, such as other anti-cancer agents, can also be incorporated into the compositions.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Solutions of the active ingredients as free base or pharmacologically acceptable salts can be prepared in water suitably mixed with surfactant, such as hydroxypropylcellulose. Dispersions also can be prepared in glycerol, liquid polyethylene glycols, mixtures thereof and in oils. Under ordinary conditions of storage and use, these preparations contain a preservative to prevent growth of microorganisms. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

An effective amount of the agents is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual.

All of these forms are generally selected to be sterile and stable under the conditions of manufacture and storage.

B. Routes of Administration

The active compounds of the present invention can advantageously be formulated for enteral administration, e.g., formulated for oral administration. The pharmaceutical forms may include sesame oil, peanut oil or aqueous propylene glycol; and sterile powders for the extemporaneous preparation of ingestible compositions, including tables, pills and capsules. Also, it is contemplated that the agents of the present invention can be provided in the form of a food additive and incorporated into a daily dietary program.

In addition to the compounds formulated for enteral administration, parenteral formulations such as intravenous or intramuscular injection are envisioned. Administration may also be nasal, buccal, rectal, vaginal or topical. Alternatively, administration may be by intradermal, subcutaneous, or intraperitoneal injection. Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the particular methods of preparation are vacuum-drying and freeze-drying techniques which yield a powder of the active ingredient plus any additional desired ingredient from a previously sterile-filtered solution thereof.

C. Combination Treatments

In one embodiment, the UIM-containing pepties may be used in conjunction with another cancer therapy, such as radiation, chemotherapy, immunotherapy, hormone therapy, toxin therapy or surgery. These compositions would be provided in a combined amount effective to kill or inhibit proliferation of the cell. This process may involve contacting the cells with the agents at the same time. This may be achieved by contacting the cell with a single composition or pharmacological formulation that includes both agents, or by contacting the cell with two distinct compositions or formulations, at the same time, wherein one composition includes UIM peptide and the other includes the second agent.

Alternatively, the UIM peptide therapy may precede or follow the other agent treatment by intervals ranging from minutes to weeks. In embodiments where the other agent and UIM peptides are applied separately to the cell, tissue or organism, one would generally ensure that a significant period of time did not expire between the time of each delivery, such that the agents would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Multiple administrations of each agent are contemplated. For example, where the UIM peptide therapy is “A” and the secondary agent or therapy is “B,” the following are contemplated:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Patients will be evaluated for neurological changes considered to be independent of tumor and graded using NCI Common Toxicity Criteria (neurotoxicity). Aside from baseline audiometric testing, repeat audiometric testing for ototoxicity is performed at the physician's discretion for patients who had evidence of hearing loss or progression of hearing loss by neurological examination. In addition, blood counts should be performed biweekly, and serum creatinine, alkaline phosphatase, bilirubin and alanine amino-transferase tested before each cycle. Doses may be modified during the course of treatment, primarily based on neutrophil and platelet counts or ototoxicity.

Chemotherapy. A variety of chemical compounds, also described as “chemotherapeutic” or “genotoxic agents,” are intended to be of use in the combined treatment methods disclosed herein. In treating cancer according to the invention, one would contact the tumor cells with an agent in addition to the expression construct. Various classes of chemotherapeutic agents are comtemplated for use with in combination with peptides of the present invention. For example, selective estrogen receptor antagonists (“SERMs”), such as Tamoxifen, 4-hydroxy Tamoxifen (Afimoxfene), Falsodex, Raloxifene, Bazedoxifene, Clomifene, Femarelle, Lasofoxifene, Ormeloxifene, and Toremifene.

Chemotherapeutic agents contemplated to be of use, include, e.g., camptothecin, actinomycin-D, and mitomycin C. The invention also encompasses the use of a combination of one or more DNA damaging agents, whether radiation-based or actual compounds, such as the use of X-rays with cisplatin or the use of cisplatin with etoposide. The agent may be prepared and used as a combined therapeutic composition, or kit, by combining it with a UIM peptide, as described above.

Heat shock protein 90 is a regulatory protein found in many eukaryotic cells. HSP90 inhibitors have been shown to be useful in the treatment of cancer. Such inhibitors include Geldanamycin, 17-(Allylamino)-17-demethoxygeldanamycin, PU-H71 and Rifabutin.

Agents that directly cross-link DNA or form adducts are also envisaged. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include Adriamycin, also known as Doxorubicin, Etoposide, Verapamil, Podophyllotoxin, and the like. Widely used in a clinical setting for the treatment of neoplasms, these compounds are administered through bolus injections intravenously at doses ranging from 25-75 mg/m² at 21 day intervals for Doxorubicin, to 35-50 mg/m² for etoposide intravenously or double the intravenous dose orally. Microtubule inhibitors, such as taxanes, also are contemplated. These molecules are diterpenes produced by the plants of the genus Taxus, and include paclitaxel and docetaxel.

Epidermal growth factor receptor inhibitors, such as Iressa, mTOR, the mammalian target of rapamycin, also known as FK506-binding protein 12-rapamycin associated protein 1 (FRAP1) is a serine/threonine protein kinase that regulates cell growth, cell proliferation, cell motility, cell survival, protein synthesis, and transcription. Rapamycin and analogs thereof (“rapalogs”) are therefore contemplated for use in combination cancer therapy in accordance with the present invention.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Radiation. Factors that cause DNA damage and have been used extensively for cancer therapy and include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells. The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

Surgery. Approximately 60% of persons with cancer will undergo surgery of some type, which includes preventative, diagnostic or staging, curative and palliative surgery. Curative surgery as a cancer treatment may be used in conjunction with other therapies, such as the treatment of the present invention, chemotherapy, radiotherapy, hormonal therapy, gene therapy, immunotherapy and/or alternative therapies. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Cytokine Therapy. Another possible combination therapy with the peptides claimed herein is TNF-α (tumor necrosis factor-alpha), a cytokine involved in systemic inflammation and a member of a group of cytokines that stimulate the acute phase reaction. The primary role of TNF is in the regulation of immune cells. TNF is also able to induce apoptotic cell death, to induce inflammation, and to inhibit tumorigenesis and viral replication.

Immunotherapy. Immunotherapy is generally defined as fostering an immune response against a tumor cell or cancer. This can take many forms, and may overlap with cytokine therapy to the extent that administered cytokines help stimulate the immune system. However, one particular immunotherapy involves the provision on anti-cancer antibodies. Where the antibodies themselves are therapeutic, this can be considered a passive immunotherapy. Examples of therapeutic antibodies include Herceptin® and Erbitux®.

Hormone Therapy. Hormone therapies are most commonly employed where a cancer has some hormonal aspect, such as breast and ovarian cancers. Unlike hormone replacement, cancer hormone therapy seeks to block the positive effect of some hormones on cancer cells, and thus are actually hormone antagonists (e.g., anti-estrogens).

Toxin Therapy. Toxins may be used to selectively kill any disease causing cell, including a tumor cell. A variety of toxins have been used for this purpose, including cholera toxin, ricin and pertussin toxin. The difficulty with use of toxins in in vivo applications is their non-selectivity, and toxicity to non-target cells. As such, schemes for selective delivery are envisioned, most commonly using tumor-homing peptides and antibodies that bind to structures not present on normal cells but found on cancer cells, or structures that are overexpressed on cancer cells as compared to normal cells.

Phenyl N-tert-butyl nitrones (PBNs). The compound phenyl N-tert-butyl nitrone (PBN) was first synthesized in the 1950's, but in 1968 it was discovered to be very useful to trap and stabilize free radicals in chemical reactions and hence it was termed a spin-trap (Janzen, 1971). Although PBN is the prototype spin-trap, several other nitrones have been synthesized and found useful to trap and characterize free radicals in chemical reactions. These spin traps were used in chemical reactions first, but in the mid-1970's they began to be used to trap free radicals in biochemical and biological systems (Floyd et al., 1977; Poyer et al., 1978). Pharmacokinetic studies have shown that PBN is readily and rapidly distributed almost equally to all tissues, has a half-life in rats of about 132 minutes and is eliminated mostly in the urine. Relatively few metabolism studies have been done, but it is known that some ring hydroxylation (primarily in the para position) of the compound occurs in the liver.

Novelli first showed that PBN could be used to protect experimental animals from septic shock (Novelli et al., 1986), and indeed this was later confirmed by other groups (Pogrebniak et al., 1992). The use of PBN and derivations as pharmacological agents began after discoveries in 1988 that showed that PBN had neuroprotective activity in experimental brain stroke models (Floyd, 1990; Floyd et al., 1996; Carney et al., 1991). These results were repeated and extended, (see Clough-Helfman et al., 1991; Cao et al., 1994; Folbergrova et al., 1995; Pahlmark et al., 1996). Others inventors have summarized the extensive neuroprotective pharmacological research effort on PBN and derivatives (Floyd, 1997; Hensley et al., 1996). In addition to neurodegenerative diseases, PBN has been shown to protect in other pathological conditions where ROS-mediated processes are involved, including diabetes and many other conditions. The mechanistic basis of why PBN and some of its derivatives are so neuroprotective in experimental stroke and several other neurodegenerative models has not been completely elucidated yet. However, it is clear that its action cannot simply be explained by its ability to trap free radicals.

The general formula for PBNs is:

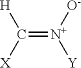

wherein:

X is phenyl or

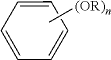

R is H,

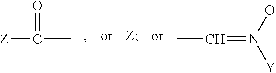

and n is a whole integer from 1 to 5; or

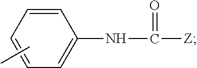

Y is a tert-butyl group that can be hydroxylated or acetylated on one or more positions; phenyl; or

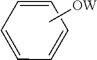

wherein W is

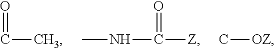

or Z; and Z is a C₁ to C₅ straight or branched alkyl group.

U.S. Pat. No. 5,569,902 (incorporated herein by reference) describes the use of nitrone free radical trapping agents for the treatment of cancer. Specifically, PBN and related compounds are described as being useful in the preparation of an anti-carcinogenic diet and the preparation of such supplemented diets. Those subjects most likely to beneficially receive the nitrones would include: (1) those having had pretumor tests indicating a high probability of the presence of tumors, (2) those exposed to very potent carcinogenic environments and their probability of tumor progression is high, and (3) to those whose genetic predisposition makes their likelihood of tumor development high.

U.S. Patent Publication 2007/0032453 (incorporated herein by reference) describes the effect of the anti-inflammatory phenyl N-tert-butyl nitrones (PBNs) on gliomas using MRI techniques. PBN itself was able to control tumor development when provided to a subject either before, at the time of or after tumor implantation. Thus, it was proposed to use PBN, and related nitrone free radical trapping agents, as therapeutic agents for gliomas.

U.S. Pat. No. 5,488,145 (incorporated herein by reference) describes 2,4-disulfonyl phenyl-tert-butyl nitrone and its pharmaceutically acceptable salts. These materials were described as useful pharmaceutical agents for oral or intravenous administration to patients suffering from acute central nervous system oxidation as occurs in a stroke or from gradual central nervous system oxidation which can exhibit itself as progressive central nervous system function loss.

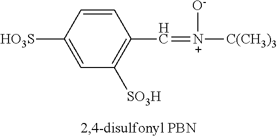

2,4-disulfonyl PBN's two sulfonate groups was expected to exhibit improved water solubility, but was also expected to exhibit poor transport across the blood/brain barrier because of its lipophobic character. However, when the present compound was made and tested in vivo, it showed an unexpected increase in efficacy as compared to PBN. This increase in efficacy occurred along with an increase in potency as compared to PBN. In direct contrast to this marked increase in potency and efficacy there was a marked and highly significant decrease in toxicity as compared to PBN.

These results were unexpected because in the general literature on structure/activity relationships within specific defined families of compounds therapeutic potency typically covaries with toxicity. Thus, most related compounds maintain their ratio of therapeutic potency to toxicity. In contrast, the compound of this invention deviates from this expected relationship when its potency increased and its toxicity decreased relative to closely related analogs.

Accordingly, in one aspect, the invention provides the PBN-disulfonyl compound and its pharmaceutically acceptable salts. In a second aspect, the invention provides intravenously- and orally-administrable pharmaceutical compositions having this compound or its salt as active ingredient.

2,4-ds PBN may exists at higher pHs in an ionized salt form:

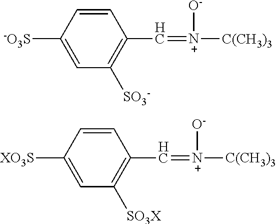

where X is a pharmaceutically acceptable cation. Most commonly, this cation is a monovalent material such as sodium, potassium or ammonium, but it can also be a multivalent alone or cation in combination with a pharmaceutically acceptable monovalent anion, for example calcium with a chloride, bromide, iodide, hydroxyl, nitrate, sulfonate, acetate, tartrate, oxalate, succinate, palmoate or the like anion; magnesium with such anions; zinc with such anions or the like. When these combinations of a polyvalent cation and a monovalent anion are illustrated in structural formulae, herein, the monovalent anion is identified as “Y.”

Among these materials, the free acid and the simple sodium, potassium or ammonium salts are most preferred with the calcium and magnesium salts also being preferred but somewhat less so.

2,4-ds PBN can be prepared by a two-step reaction sequence. In the first step, commercially available tertiary butyl nitrate (2-methyl-2-nitropropane) is converted to the corresponding n-hydroxyl amine using a suitable catalyst such as an activated zinc/acetic acid catalyst or an aluminum/mercury amalgam catalyst. This reaction can be carried out in 0.5 to 12 hours and especially about 2 to 6 hours or so at a temperature of about 15-100° C. in a liquid reaction medium such as alcohol/water mixture in the case of the zinc catalyst or an ether/water mixture in the case of the aluminum amalgam catalyst.

In the second step, the freshly formed hydroxylamine is reacted with 4-formyl-1,3-benzenedisulfonic acid, typically with a slight excess of the amine being used. This reaction can be carried out at similar temperature conditions. This reaction is generally complete in 10 to 24 hours.

The product so formed is the free acid and is characterized by a molecular weight of 89 g/mole. It is a white powdery material which decomposes upon heating. It is characterized by a solubility in water of greater than 1 gram/ml and a ¹H NMR spectrum in D₂ O of 8.048 ppm (dd, 8.4, 1.7 Hz); 8.836 ppm (d, 8.4 Hz); 8.839 ppm (d, 1.7 Hz); 8.774 ppm (s).

The various salts can be easily formed by admixing the free acid in aqueous medium with two equivalents of the appropriate base, for example, KOH for the potassium salt, and the like.

One synthesis is based on the work by Hinton and Janzen (1992). It involves the condensation of an aldehyde with a hydroxylamine. The hydroxylamine is unstable and is prepared fresh on the day of use using an activated zinc catalyst. The synthesis is as follows.

TABLE 1
Prerequisite Chemicals

```
  1. 95% Ethanol
  2. 2-Methyl-2-nitropropane
  3. Zinc dust
  4. Glacial acetic acid
  5. Diethyl ether
  6. Saturated sodium chloride
  7. Magnesium Sulfate, Anhydrous solid
  8. 4-Formyl-1,3-benzenesulfonic acid
(MW 310.21 g/mole), disodium salt, hydrate
  9. Methanol
10. Dichloromethane
```

TABLE 2
Preparation of N-t-Butylhydroxylamine

```
  1. A 500 mL three neck round bottom flask is equipped with a magnetic
stir bar, thermometer adapter, thermometer, and addition funnel.
  2. 95% ethanol (350 mL) was added to the flask and cooled to 10° C.
in an ice bath.
  3. 2-Methyl-2-nitropropane (6.18 g, 0.060 mole), and zinc dust
(5.89 g, 0.090 mole) were added in single portions.
  4. Glacial acetic acid (10.8 g, 0.180 mole) was placed in the addition
funnel and added dropwise at such a rate with vigorous stirring to maintain
the temperature below 15° C.
  5. The ice bath was removed and mixture was stirred for 3 hrs at
room temperature.
  6. The solvent was stripped from the mixture, leaving
t-butylhydroxylamine, zinc acetate, and water.
  7. Dichloromethane (50 mL) was added and the mixture filtered
through a Buchner funnel.
  8. The zinc acetate cake left on the filter paper was washed
with 2X 25 mL dichloromethane.
  9 Water was separated from the filtrate in a separatory funnel and the
organic layer dried over magnesium sulfate.
10. The magnesium sulfate was removed by filtering through fluted
filter paper, then dichloromethane stripped off by rotary evaporation.
11. The product (100% yield = 5.34 g), viscous liquid, was
dissolved in methanol (50 mL) for use below.
```

TABLE 3
Preparation of 2,4-disulfonylphenyl-N-t-butylnitrone

```
  1. A 3 -neck 250 ml round bottom flask was set up with a stir bar,
a gas dispersion tube, an addition funnel, and a Friedrichs condenser
cooled with recirculating ice water.
  2. To the flask were added 200 mL of methanol,
4-formyl-1,3-benzenedisulfonic acid (9.31 g, 30 mmoles) and
N-t-butylhydroxylamine (25 mL of the methanol solution
from part A, 30 mmoles theoretical).
  3. The reaction was heated to reflux with a heating mantle while
bubbling the reaction with nitrogen with stirring.
  4. The mixture was refluxed for 2 hours.
  5. The remainder of hydroxylamine from above was added.
  6. Refluxing was continued with nitrogen bubbling for at least
18 hours, but not more than 24 hours.
  7. The hot reaction mixture was filtered on a Buchner funnel,
and the solid washed with hot methanol.
  8. The methanol was stripped off by rotary evaporation to a yellow,
viscous oil.
  9. Hot 1:1 ethanol:acetone (200 mL) was added and the mixture
heated to dissolve the oil.
10. The solution was cooled to crystallize the product.
11. The product was collected on a Buchner funnel and dried under
vacuum overnight.
12. The reaction typically gives 75% yield of I, a white powder.
```

Other methods of synthesis are disclosed in the prior art as well.

### 5. EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute specifically contemplated modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Generation of conditional Epn1fl/fl mice and EC-DKO or EC-iDKO mice. The inventors recently reported a strategy for generation of an epsins 1 and 2 global double knockout (DKO) mouse model (Chen et al., 2009). The inventors used a similar strategy with modifications to create conditional knockout of epsin 1 (Epn1f/f mice). Epn1fl/fl mice were mated with Epn2−/− to generate Epn1fl/fl, Epn2−/− mice. Endothelial cell-specific DKO mice (EC-DKO) were obtained by crossing Epn1fl/fl, Epn2−/− mice with Tie2 Cre deleter mice, which specifically inactivate epsin 1 gene in endothelial and hematopoietic cells. Tamoxifen inducible endothelial cell-specific DKO mice (EC-iDKO) were obtained by crossing Epn1fl/fl, Epn2−/− mice with VEcad-ERT2 Cre deleter mice, which specifically inactivate epsin 1 gene in endothelial cells upon tamoxifen administration.

Immunohistochemistry and immunofluorescence of tissue samples. Immunohistochemistry and immunofluorescence were performed as described with modifications (Chen et al., 1998; Chen et al., 2009).
E9/10 Hind Brain: Embryos were harvested at E9 or E10 and fixed. Hindbrain was harvested and processed for staining with anti-CD31 and donkey anti-rat Alex Fluor 488 secondary antibody.
P6 Skin: Wild-type or EC-iDKO pups were injected intraperitoneally with 5 mg/kg (body weight) of 4-hydroxytamoxifen (10 mg/ml of 4-Hydroxytamoxifen resuspended in 10% of ethanol and 90% of DMSO) per day from postnatal day 1 (P1) to P3. Pups were euthanized at P6 and skin from abdomen was harvested and processed for whole mount staining with anti-CD31 and donkey anti-rat Alexa Fluor 488 secondary antibody.
P6 Retina: Wild-type or EC-iDKO Pups were injected with 4-hydroxytamoxifen as described above per day from postnatal day 2 (P2) to P4. P6 pups were euthanized and whole eyes harvested. Retinas were harvested and processed for whole mount staining with biotinylated isolectin B4 and Streptavidin Alexa Fluor 488 secondary Ab.
Intraocular injection: Wild-type or EC-iDKO Pups were injected with 4-hydroxytamoxifen as described above per day from postnatal day 2 (P2) to P4. Intraocular injection of VEGFR-2 antibodies, inhibitors to FGFR or PDGFR or saline to P6 retina was performed as previously described (Gerhardt et al., 2003).

Antibodies and reagents. Polyclonal rabbit antibodies for epsin 1 and epsin 2 were obtained as previously described (Rosenthal et al., 1999; Chen et al., 1998), anti-EEA1, anti-dynamin 2, goat anti-epsin 1 and mouse anti-VEGFR-2 were obtained from Santa Cruz; anti-CD31 and anti-LAMP1 from BD; anti-clathrin heavy chain from Affinity BioReagents; anti-CD63 from Chemicon; Rabbit anti-VEGFR-2, VEGFR-1, VEGFR-3, antiphospho-VEGFR-2 (pY1175), anti-PLCfÁ, anti-phospho-PLCfÁ, anti-ERK, and anti-phospho-ERK from Cell Signaling Technology; anti-phospho-VEGFR-2 (pY1054/1059) from Millipore. VEGFA, FGF and PDGF were from R&D systems. Biotinylated isolectin B4 was from Vector Labs. 4-hydroxytamoxifen and human fibronectin were from Sigma. γ-secretase, VEGFR inhibitor (SU1498), FGFR inhibitor and PDGFR inhibitor were obtained from Calbiochem. Matrigel was from BD. Dynasore was from Santa Cruz or Tocris.

Plasmids and transfection. Mammalian expression plasmids for epsin 1, VEGFR-2 and their mutants were described previously (Chen and De Camilli, 2005; Zhang et al., 2008). A ubiquitination-deficient VEGFR-2 mutant was created by mutating (Chen and De Camilli, 2005) conserved lysine residues among human, mouse, rat, chicken and zebrafish to arginines in the cytoplasmic domain of VEGFR-2 using QuikChangeR Site-Directed Mutagenesis Kit (Stratagene). Notch NICD expression plasmid is a kind gift from Dr. Michael Potente (Frankfurt, Germany). MECs were transfected with NICD, epsin 1 or epsin 1f¢UIM constructs using Amaxa Nucleofector device (Lonza) according to the manufacture's protocol.

Cell culture. HUVEC and BAEC were purchased from Lonza and cultured according to the manufacture's protocol. Cells were used between passage 2 and 5. HEK 293T cells were transfected with Lipofectamine 2000 according to the manufacture's instructions. Primary mouse endothelial cell (MECs) isolation from brain was performed as we described previously (Zhang et al., 2008). MECs isolated from wild-type and EC-iDKO mice were treated with 5 μM of 4-hydroxytamoxifen dissolved in ethanol for two days at 37° C. followed by incubation for additional two days without 4-hydroxytamoxifen. Deletion of epsin 1 was confirmed by both western blot and immunohistochemistry using epsin 1 antibodies. Freshly isolated primary MECs were used for all experiments without any further passages.

RNA interference. HUVEC were transfected with siRNA duplexes of scrambled or human epsin 1 (UGCUCUUCUCGGCUCAAACUAAGGG) (SEQ ID NO:1) and epsin 2 (AAAUCCAACAGCGUAGUCUGCUGUG) (SEQ ID NO:2), clathrin heavy chain (CGCGGUUACUUGAGAUGAACCUUAU) (SEQ ID NO:3), dynamin 2 (GGAUAUUGAGGGCAAGAAG) (SEQ ID NO:4) and (GCGAAUCGUCACCACUUAC) (SEQ ID NO:5) using AmbionR SilencerR Select Pre-designed siRNAs (Invitrogen) by Oligofectamine or RNAiMAX according to the manufacture's instructions. At 48-72 h post transfection, cells were processed for biochemical analysis or wound and network formation assays.

Immunoprecipitation and western blot analyses. For Sequential immunoprecipitation (IP), transfected 293T cells were lysed with RIPA Buffer (1% Triton X-100/0.1% SDS/0.5% sodium deoxycholic acid/5 mM tetrasodium pyrophosphate/50 mM sodium fluoride/5 mM EDTA/150 mM NaCl/25 mM Tris, pH 7.5/5 mM Na₃VO₄/5 mM Nethylmaleimide and protease inhibitor cocktail). Cell lysates were precleared with mouse IgG and protein G beads for 2 h at 4° C. followed by incubation with anti-Flag for 4 h at 4° C. Precipitated proteins were eluted from beads using 2% SDS in 50 mM Tris, pH 7.5 and diluted 1:20 with RIPA Buffer followed by anti-VEGFR-2 immunoprecipitation and western blotting. For immunoprecipitation using BAEC cells, 90% confluent BAEC were starved for 24 h at 37° C. with DMEM. Cells were stimulated with 50 ng/ml of VEGF-A for 0, 2, 5, 15, 30 m and harvested using RIPA buffer. Cell lysates were precleared with goat IgG and protein G sepharose beads at 4° C. for 2 h followed by incubation with goat anti-epsin 1 as described above. For negative controls, goat IgG was added instead of goat anti-epsin 1 and immunoprecipitation was carried out using cell lysate prepared from cells exposed to 50 ng/ml of VEGF-A for 2 m. For VEGF, FGF and PDGF signaling assays, MECs that had been starved 16 h in serum-free medium were treated with 50 ng/ml of VEGF-A, 25 ng/ml FGF and 25 ng/ml PDGF for 0, 5 or 15 m at 37° C. and processed for western blotting directly. For VEGF signaling assays with Dynasore, MECs that had been starved 16 h in serum free medium were pretreated with 40 or 80 μM of Dynasore for 2 h before adding 50 ng/ml of VEGFA for the time points indicated at 37° C. and processed for western blotting directly.

Endocytosis assays. ELISA of cell surface VEGFR-2. MECs were starved overnight before treated with 50 ng/ml of VEGF-A for 0, 10 or 20 m at 37° C. to allow internalization of cell surface VEGFR-2. At the end point of treatment, cells were incubated with 1 mM EZ-Link Sulfo-NHS-LC-Biotin on ice for 30 m and washed with 50 mM glycine followed cell lysis with RIPA buffer. Mouse anti-VEGFR-2 monoclonal antibody directed against the extracellular domain of VEGFR-2 (0.5 μg/well) was added to cell lysates and incubate for 16 h at 4° C. followed by incubation with 0.1 μg/ml streptavidin-HRP for 1 h at 37° C. ABTs Peroxidase Substrate solution was added followed by absorbance measuring at 405 nm with a micro-plate reader.

Internalization of biotinylated VEGFR-2. MECs were starved overnight and incubated with 1 mM EZ-Link Sulfo-NHS—S—S-Biotin dissolved in cold PBS at 40 C for 30 m. Cells were then changed to warm media with 50 ng/ml VEGF-A and incubated at 37° C. for 0, 10 or 20 m. Remaining surface biotin attached to uninternalized plasma membrane proteins was then removed by incubation with iced cold PBS/50 mM glycine for 30 m at 4° C. Cells were lysed in RIPA buffer and processed for streptavidin bead pull down. 30% of the pull down from lysates prepared from cells that were not treated with glycine was loaded for western blotting. Endocytosed VEGFR-2 was visualized by western blotting using anti-VEGFR-2 antibodies and quantified by NIH Image 1.60.

Wound and network/tube formation assays. Monolayer EC wound assay. Monolayer EC wound assays were performed as described (Zhang et al., 2008). EC network or tube formation. EC network/tube formation in Matrigel was performed as described (Zhang et al., 2008).

Immunofluoresence imaging of cells. Immunofluorescence was performed as described with modifications (Chen et al., 1998; Chen et al., 2009). Biotinylation of VEGF-A and confocal imaging. VEGF-A was labeled with Biotin (EZ-LinkR Micro

Sulfo-NHS-LC Biotinylation Kit) according to the manufacture's instructions. HUVEC, MECs or 293T cells were plated on coverslips precoated with 0.2% gelatin and grown to 75% confluency. Cells were serum starved overnight and incubated with 100 ng/ml of biotinylated VEGF-A for 30 m at 4° C. Streptavidin Alexa Fluor 488 was added and incubated for another 30 m at 4° C. Cells were then shifted to 37° C. for 1, 2, 5, 10, 20 and 30 m to allow internalization of VEGFR-2. At the end of 10, 20 and 30 m, WT MECs but not DKO MECs were acid washed (0.15 M NaCl, 0.5M acetic acid at pH4.5) for 5 m at 4° C. to remove cell surface bound biotinylated VEGF-A/Streptavidin Alexa Fluor 488, then fixed with 1% formaldehyde in PBS for colocalization analysis. Cells were permeabilized, incubated with primary goat anti-epsin 1, goat anti-EEA1, mouse anti-CD63 or LAMP1 antibodies followed by incubation with fluorescent secondary antibodies. Cells were then washed and mounted, and photomicrographs were obtained using an Olympus IX81 Spinning Disc Confocal Microscope with an Olympus plan Apo Chromat 60× objective and Hamamatsu Orca-R2 Monochrome Digital Camera C1D600.

VEGFR-2 antibody staining and confocal imaging. HUVEC or MECs were stimulated with 50 ng/ml of VEGF-A at 37° C. as described above. Cells were fixed, processed for immunostaining with rabbit anti-VEGFR-2 and goat anti-epsin 1 or rabbit anti-phospho-VEGFR-2, along with goat anti-EEA1 or mouse anti-CD63 antibodies for 2 h at RT, then incubated with fluorescent secondary antibodies for 1 h at RT. Cells were washed and mounted, and photomicrographs were obtained as described above.

Statistical analysis. Data were analyzed by the student's t test or ANOVA, where appropriate. The Wilcoxon signed rank test was used to compare data that did not satisfy the student's t test or ANOVA.

Tumor implantation. To induce postnatal deletion of endothelial epsin 1, the inventors administered 4-hydroxytamoxifen (50 μg per 30 g of body weight) by IP injection into six-week-old WT or epsin 1fl/fl/Cre-ERT2/epsin 2−/− mice. Injections were performed once per day for five consecutive days, followed by a 5-7 day resting period to obtain WT and EC-iDKO mice. To assess tumor growth, the inventors implanted Lewis Lung Carcinoma cells (LLC cells, ATCC, 1×10⁶ cell/tumor) in EC-iDKO and WT mice. They estimated the time of tumor appearance and monitored the tumor growth in two groups of mice by measuring tumor size with digital calipers. The inventors recognized tumors more than 2 mm in diameter as positive and calculated tumor volume based on the formula 0.5326 (length [mm]×width [mm]²).

BrdU labeling. BrdU labeling of Mouse Endothelial Cells. WT and DKO MECs were grown in a 48-well plate until they reached 50% percent confluency. Cells were starved overnight and stimulated with growth factors or growth factors plus inhibitors for 6 h. BrdU labeling and detection kit (Roche) was then used to label the proliferating cells. Briefly, cells were incubated with BrdU labeling medium (1:500 diluted in medium) for 3 h. Cells were washed three times with wash buffer and fixed with ethanol for 20 min at −20° C. followed by washing the cells three times with wash buffer and incubated with 6M HCl/0.1% Triton for 30 minutes at room temperature. This was followed by six washes with PBS/0.1% Triton. Cells were blocked with PBS/0.3% Triton/3% BSA/3% donkey serum for 30 min at room temperature followed by incubation with anti-BrdU working solution for 30 minutes at 37° C. After three washes with wash buffer cells were again incubated with donkey anti-mouse Ig-fluorescein for 30 minutes at 37° C. After washing the cells three times with wash buffer they were stained with DAPI and visualized using Olympus Fluorescent microscope. Percentage of proliferating cells was calculated based on the ratio of BrdU-positive cells versus DAPI-positive cells.

In vivo BrdU labeling. Preganant WT or EC-iDKO female mice were injected intraperitoneally with 5 mg/kg (body weight) of 4-hydroxytamoxifen at E13. Five days later, 100 μl of BrdU labeling reagent (BrdU Labeling and Detection kit 1, Roche) was intravenously injected for 90 min. Mice were euthanized and E18 embryo's were harvested and fixed in 4% PFA. Intestinal samples were processed for whole mount staining with anti-CD31 and anti-BrdU antibodies.

Receptor tyrosine kinase inhibitors injection. Wild-type or EC-iDKO pups were injected intraperitoneally with 5 mg/kg (body weight) of 4-hydroxytamoxifen per day from postnatal day 1 (P 1) to P 3. WT and EC-iDKO P6 pups were given either mock injection (DMSO) or inhibitor injection. VEGF, FGF or PDGF receptor tyrosine kinase inhibitors (resuspended in DMSO) were injected intraperitoneally at a concentration of 30 mg/kg of body weight. Some pups were also given Intraocular injections of inhibitors at 5 μg/eye. After 7 h of injection pups were euthanized and whole eyes were harvested and fixed in 4% PFA for 1 h at RT or 4° C. overnight. Retinas were harvested and fixed in 4% PFA for 30-60 min at RT and processed for whole mount staining with isolectin B4. For western blot analysis, skin and heart tissues were collected from WT P6 pups injected either with DMSO or inhibitors and analyzed for phospho-Akt and total Akt.

RT-PCR. Total RNA was extracted from WT or EC-DKO embryos or MECs with the Trizol Reagent (Invitrogen). One μg total RNA was treated with 1 unit RNase-free DNase I (Invitrogen) to eliminate genomic DNA. The first strand cDNA was synthesized by using the SuperScript III First-Strand Synthesis SuperMix (Invitrogen). An aliquot of 1 μl of the product was subjected to PCR reaction using gene-specific primer pairs:

Hes-1
(SEQ ID NO: 6)
(5′~ACACCGGACAAACCAAAGAC~3′,
(SEQ ID NO: 7)
1 5′~GTCACCTCGTTCATGCACTC~3′);
Hey1
(SEQ ID NO: 8)
(5′~CATGAAGAGAGCTCACC~3′,
(SEQ ID NO: 9)
5′~AATGTGTCCGAGGCCAC~3′);
Hey2
(SEQ ID NO: 10)
(5′~GACAACTACCTCTCAGATTATGGC~3′,
(SEQ ID NO: 11)
5′~CGGGAGCATGGGAAAAGC~3′);
HeyL
(SEQ ID NO: 12)
(5′~GGTCCCCACTGCCTTTGAGA~3′,
(SEQ ID NO: 13)
5′~AGGATGGCGAGCTGACTGTTC~3′);
Beta-Actin
(SEQ ID NO: 14)
(5′~GACGGCCAGGTCATCACTAT~3′,
(SEQ ID NO: 15)
5′~ACATCTGCTGGAAGGTGGAC~3′).

In vivo studies. Melanoma model. Mice at the age of ˜10 wks old were skin-implanted melanoma cell line (ATCC) at 0.25 million cells. After 14 days, tumors develop at average size of 3×4 mm. Treatment involved starting local injection of UIM peptide at 15 day post-inoculation of melanoma cancer cells, followed by daily injection at the dosage of 25 μg/tumor for 10 consecutive days. Tumor size was recorded every other day. Tumor analysis included growth rate; tumor size; angiogenesis; blood vessel perfusion; hypoxia and apoptosis.

Lewis lung carcinoma model. Mice at the age of ˜10 wks old were skin-implanted a Lewis Lung carcinoma cancer cell line (ATCC) at 0.75 million cells/animal. After 3 days of implantation, intravenous injection of UIM D-isomer peptide was started at 200 μg/mouse daily. Tumor initiation in UIM-treated mice was delayed 2 days versus PBS; n=5.

Prostate cancer model. TRAMP mice harboring SV40 large T antigen specifically expressed in prostate epithelial cells develop prostate cancer around 26 weeks. These mice were injected (IP) with UIM peptide at 200 μg/mouse twice per week for five weeks; n=6-8.

Glioma model. GL6 glioma brain tumors were generated in mice at the age of ˜10 wks old by implanting GL6 cancer cell line (ATCC) cells at 1 million cells/animal in the right lobe of brain. After 7 days of implantation, intravenous injection of UIM peptide was started at 200 μg/mouse daily. Tumor growth was monitored every two or three days by MRI; n=6-8.

### Example 2

### Results

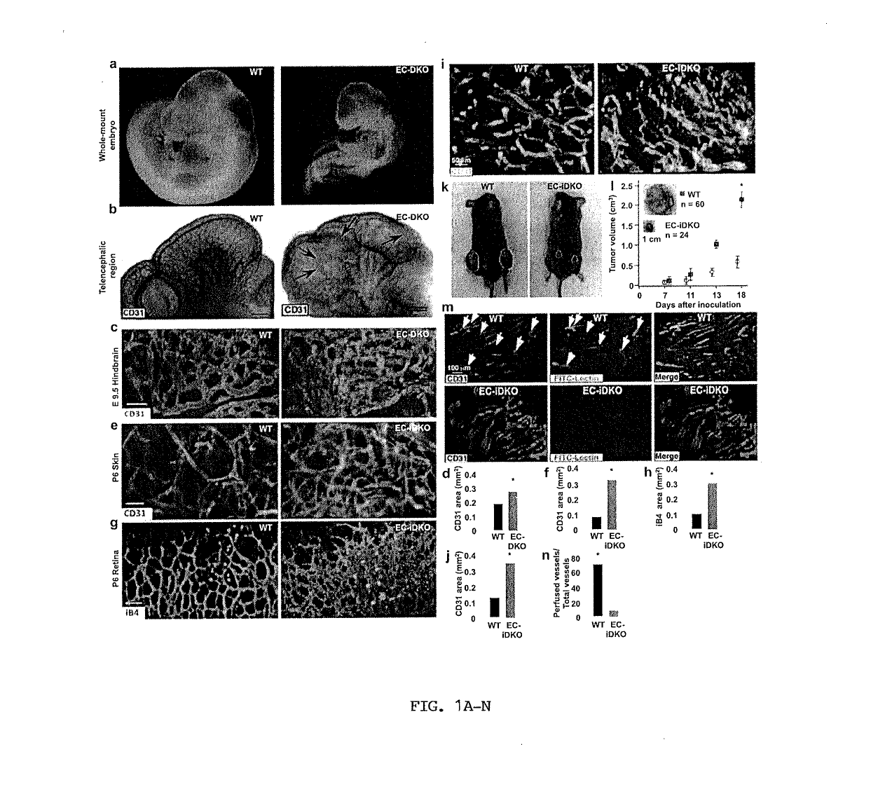

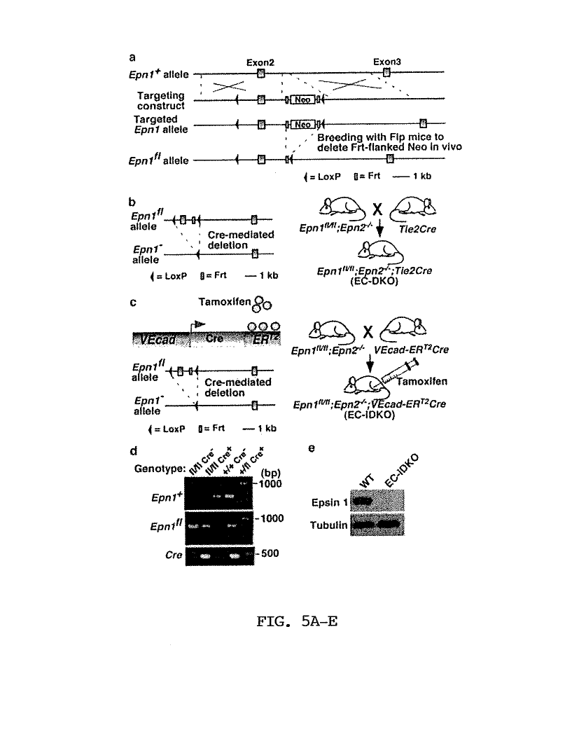

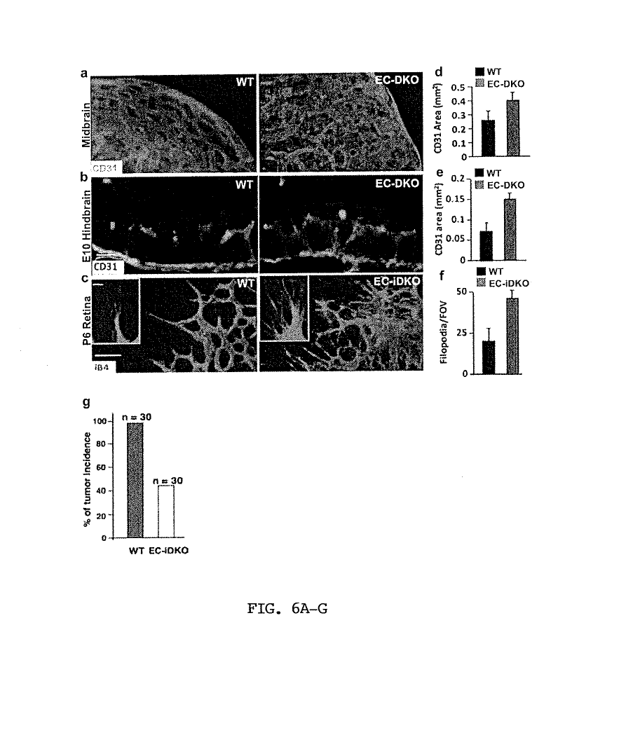

To investigate the scope of regulation by epsins in embryonic and postnatal vascular development and angiogenesis, the inventors examined a range of angiogenic tissues (embryo, dermis, retina and tumor) and different ages (E9.5 through adult) using mouse models that selectively lack epsins in endothelial cells (EC). They first created a constitutive endothelial cell specific epsin DKO (EC-DKO) by crossing conditional epsin DKO mice (Epn1fl/fl, Epn2−/−), which are indistinguishable from wild-type (WT) mice due to functional redundancy of epsin 1 and 2, to a Cre deleter strain expressing Cre recombinase from the Tie-2 promoter only in endothelial cells and cells of hematopoietic cell lineages (Kisanuki et al., 2001) (FIGS. 5A-B). The inventors observed normal hematopoietic development as evident by normal blood cells formation in EC-DKOs (not shown); however, ECDKOs die in utero around E11, at a date that is later than global DKOs which are around E9.5-E10, indicating that constitutive loss of epsins in the whole organism results in more severe phenotype than EC-specific deletion of epsins (FIG. 1A). CD31, an endothelial marker staining of the head region of EC-DKO embryos revealed prominent vascular defects with disordered vasculature (FIG. 1B) and an increased number of vessels (FIG. 6A). This is not phenocopied by global DKOs, suggesting that this phenotype reflects more precisely the loss of epsins in ECs. The inventors also observed increased angiogenesis measured by a more disorganized and denser vascular network in a whole-mount hindbrain of E9.5 EC-DKO embryos (FIGS. 1C-D) and the formation of a more fully elaborated subventricular vascular plexus in the cross section of hindbrains of E10 EC-DKO embryos (FIG. 6B).

To examine whether postnatal angiogenesis is also affected by loss of endothelial epsins, the inventors created an inducible EC-DKO (EC-iDKO) by crossing Epn1fl/fl, Epn2−/− to VEcad-ERT2 Cre deleter mice that specifically inactivate the epsin gene in EC upon tamoxifen administration (Monvoisin et al., 2006) (FIGS. 5A, 5C). Despite that postnatal angiogenesis in adults is relatively quiescent under physiological condition, it is quite active in young animals, particularly in organs undergoing rapid remodeling including skin and retina. To postnatally induce epsin 1 deletion, the inventors systemically administrated tamoxifen in young WT or EC-iDKO mice. Although, they observed no obvious gross difference between these two groups of mice at P6, a striking increase in blood vessel formation visualized by CD31 staining was evident in postnatal mouse dorsal skin isolated from EC-iDKO compared to WT (FIGS. 1E-F). Furthermore, dramatic increases in vascular networks and vascular sprouts were apparent in P6 retina of ECiDKO mice (FIGS. 1G-H and FIG. 6C), suggesting that epsins are important regulators of embryonicand postnatal angiogenesis.

Because many of the mechanisms controlling normal blood vessel formation in development also operate under pathogenic conditions, the inventors explored whether loss of endothelial epsins also affects tumor angiogenesis which is indispensable for tumor expansion. Adult WT or EC-iDKO mice were generated by tamoxifen administration. They did not observe gross abnormalities in endothelial epsin-deficient adult mice. WT or EC-iDKO mice were subcutaneously implanted with mouse Lewis Lung Carcinoma (LLC) cells to initiate tumor growth. They observed elevated tumor angiogenesis measured by increased but highly disorganized tumor vasculature as revealed by CD31 staining (FIGS. 1I-J). Paradoxically, this enhanced tumor angiogenesis led to smaller tumors with reduced growth rate (FIGS. 1K-L) and fewer tumors (FIG. 6D) developed in EC-iDKO mice. The inventors hypothesize that this elevated tumor angiogenesis may produce non-functional tumor vessels, affecting efficient blood flow. To test this, they intravenously injected fluorescein isothiocyanate (FITC)-conjugated lectin, a tracer that has been extensively used to measure perfusion ability of vessels, into tumor-bearing EC-iDKO and WT mice. Substantial intravascular FITC-lectin labeling was detected in WT but not in EC-iDKO tumor vessels costained with CD31 (FIGS. 1M-N), indicating that loss of endothelial epsins causes dysfunctional tumor vessels which limits blood flow, oxygen and nutrients supply to the tumor, and hence tumor resistance phenotype.

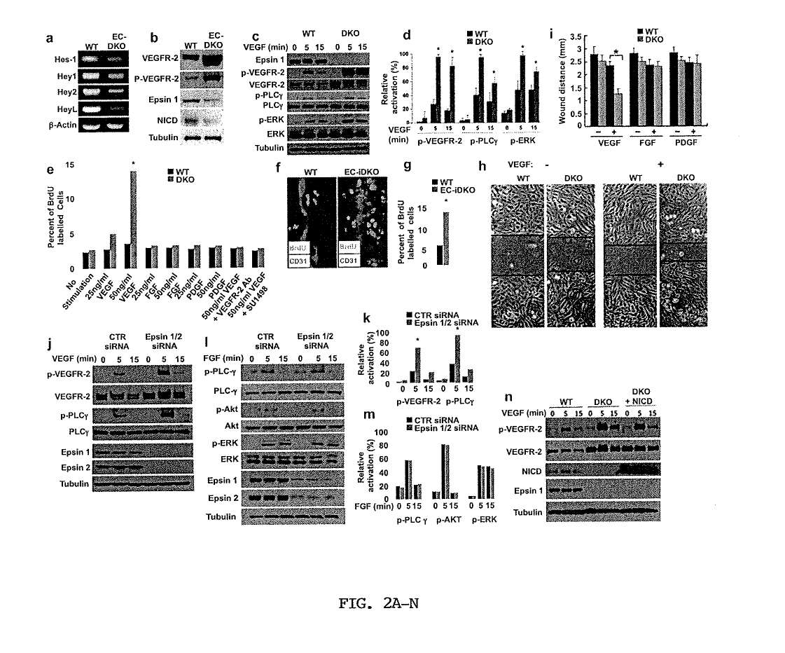

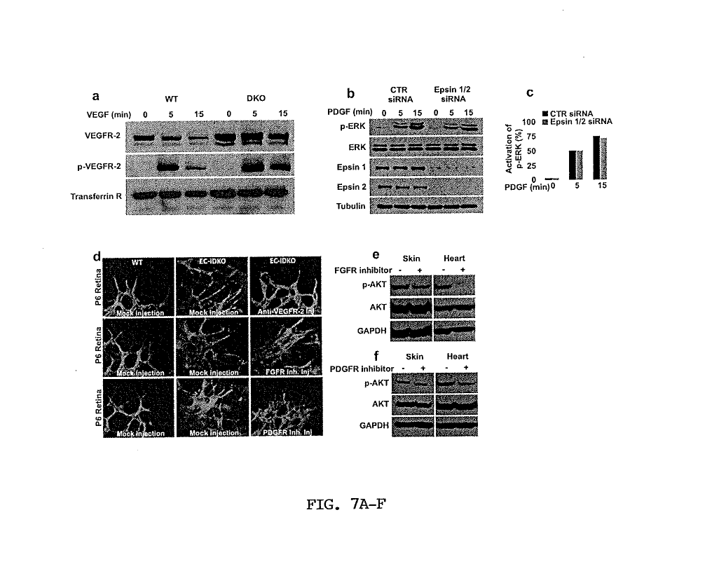

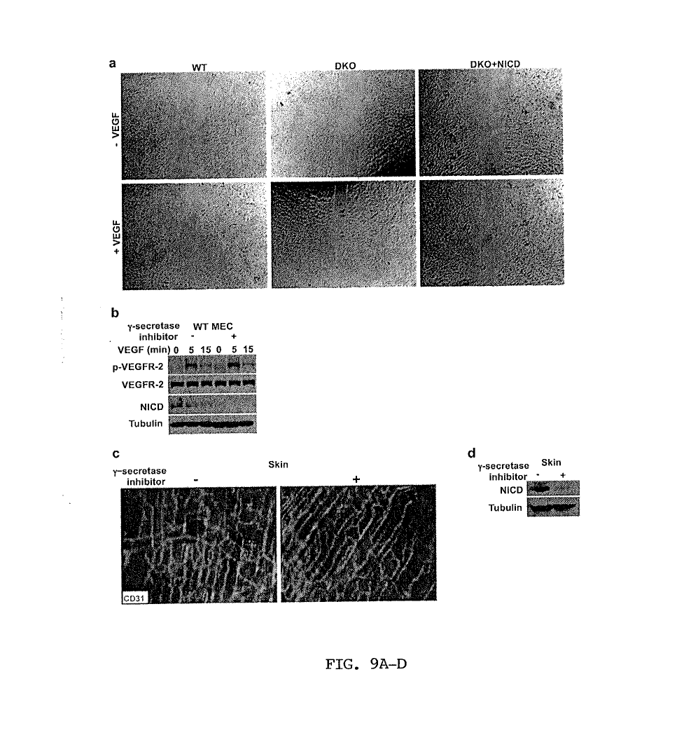

Based on the defect of Notch signaling observed in global DKO mice22 and on the established role of Notch signaling on vascular development and tumor angiogenesis (Weinstein and Lawson, 2002; Jakobsson et al., 2009; Thurston and Kitajewski, 2008; Hellstrom et al., 2007; Williams et al., 2006; Phng and Gerhardt, 2009; Suchting et al., 2007; Hellstrom et al., 2007; Phng et al., 2009), the increased angiogenesis by loss of endothelial epsins might be resulted from defective Notch signaling. Indeed, the inventors observed reduced expression of the active form of Notch (NICD) (FIG. 2B) and Notch downstream targets, Hesl, Hey1, Hey2 and HeyL in EC-DKO embryos (FIG. 2A). This elevated angiogenesis should also reflect enhanced VEGF signaling given a negative regulatory role of Notch on VEGF function (Hellstrom et al., 2007; Williams et al., 2006; Ridgway et al., 2006; Noguera-Troise et al., 2006; Phng and Gerhardt, 2009; Thurston et al., 2007; Suchting et al., 2007; Hellstrom et al., 2007). The inventors focused on VEGFR-2 among all VEGF receptor family members because of its fundamental role in angiogenesis. Increased VEGFR-2 expression and enhanced phosphorylation of VEGFR-2 were observed in EC-DKO embryos (FIG. 2B). To dissect VEGF-VEGFR-2 signaling, they isolated mouse primary endothelial cells (MECs) (Zhang et al., 2008) from WT or EC-iDKO mice and epsin was then ablated from DKO MECs by addition of tamoxifen in culturing medium. Compared to WT MECs, NICD production was decreased (FIG. 2N and FIG. 9B), however, VEGF signaling was dramatically increased in DKO MECs measured by the elevated VEGFR-2 cell surface expression (FIG. 7A), total level of VEGFR-2 (FIG. 2C), and augmented phosphorylation of VEGFR-2, and its downstream signaling molecules PLC-γ and ERK upon VEGF stimulation (FIGS. 2C-D). This increase in VEGF signaling was not due to elevated VEGF production in DKO MECs because the inventors did not observe increased level of VEGF in DKO MECs compared to WT MECs (not shown).

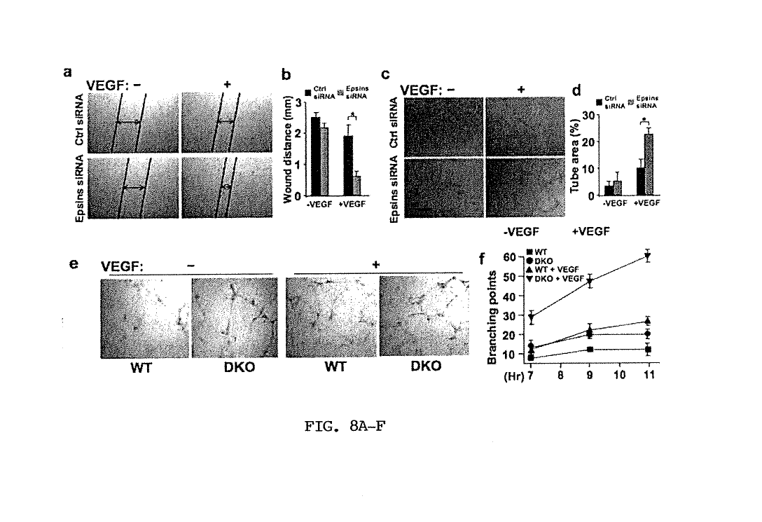

To test whether other angiogenic signaling pathways might also be affected by loss of epsins, the inventors examined the proliferation of DKO MECs in the presence of VEGF, fibroblast growth factor (FGF) and platelet-derived growth factor (PDGF). VEGF but neither FGF nor PDGF stimulation greatly enhanced the proliferation of DKO MECs relative to WT MECs measured by BrdU labeling, which was abrogated by the inhibition of VEGFR-2 (FIG. 2E). They also observed enhanced proliferation of ECs in vivo in EC-iDKO mice in which the ablation of epsin was triggered by IP injection of tamoxifen (FIGS. 2F-G). Similarly, VEGF but neither FGF nor PDGF stimulation markedly increased proliferation and migration of DKO MECs (FIGS. 2H-I) and epsin-deficient primary human umbilical cord vein endothelial cells (HUVEC) measured by wound closure and network formation assays (FIG. 8). Likewise, siRNA-mediated knockdown of epsins in HUVEC caused augmented VEGF (FIGS. 2J-K) but not FGF (FIGS. 2L-M) and PDGF (FIGS. 7B-C) signaling. Furthermore, inhibition of VEGF but not FGF or PDGF signaling blunted elevated retina angiogenesis in EC-iDKO (FIGS. 7D-F). Collectively, these data suggest that loss of endothelial epsins specifically affects VEGF signaling pathway but not other pathways implicated in angiogenesis.

Given that epsins are major clathrin adaptors that mediate endocytosis of ubiquitinated proteins, and that VEGF induces endocytosis of VEGFR-2, promoting its lysosomal degradation and thus attenuating its signalling (Haglund et al., 2003; Hicke, 1997; Murdaca et al., 2004; Duval et al., 2003; Ewan et al., 2006), the inventors postulate that this striking increase in VEGFR-2 signaling is not just a consequence of deficient Notch signaling, but rather a combinatory effect of lack of Notch activation and impaired endocytosis and degradation of VEGFR-2 by loss of endothelial epsins. To this end, they reconstituted DKO MECs with NICD to rescue defective Notch signaling and examined whether restoring Notch signaling can suppress enhanced VEGF signaling.

Surprisingly, restoring Notch signaling only partially suppressed elevated VEGF signaling (FIG. 2N) and increased proliferation and migration of DKO MECs measured by wound closure assay (FIG. 9A). Conversely, blocking Notch signaling in WT MECs by incubation with γ-secretase inhibitor did not significantly increase VEGF-induced VEGFR-2 phosphorylation (FIG. 9B) Similarly, blocking Notch signaling in vivo by γ-secretase inhibitor injection did not produce marked increase in skin angiogenesis that was observed in EC-iDKO (FIGS. 9C-D), suggesting that deficient Notch signaling only counts for a part of the increase in VEGF signaling in DKO MECs and angiogenic defects observed in EC-iDKO. The inventors hypothesize that loss of adaptor function of epsins in VEGFR-2 endocytosis is likely responsible for the rest of increase in VEGF signaling and angiogenesis.

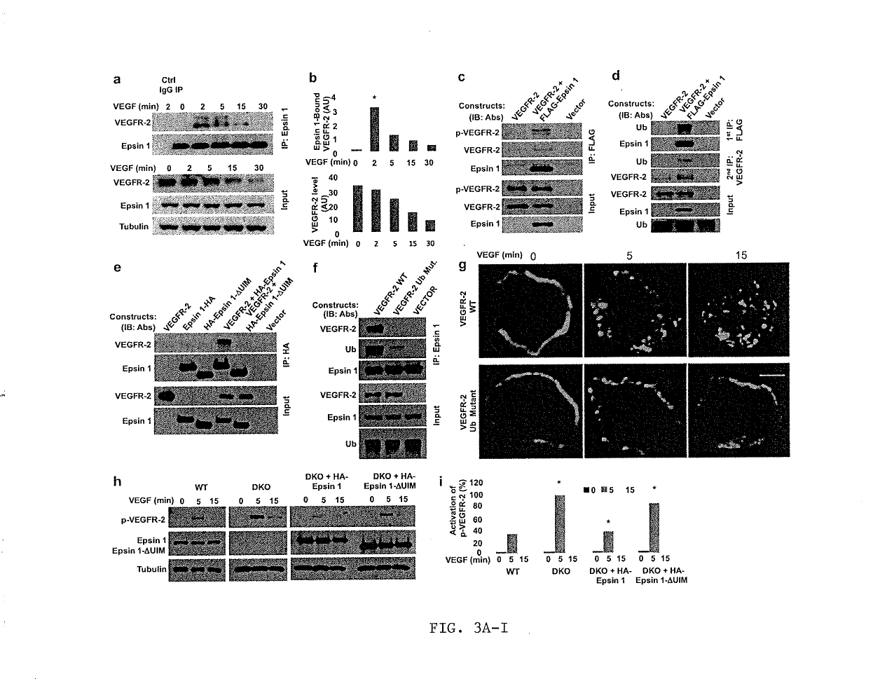

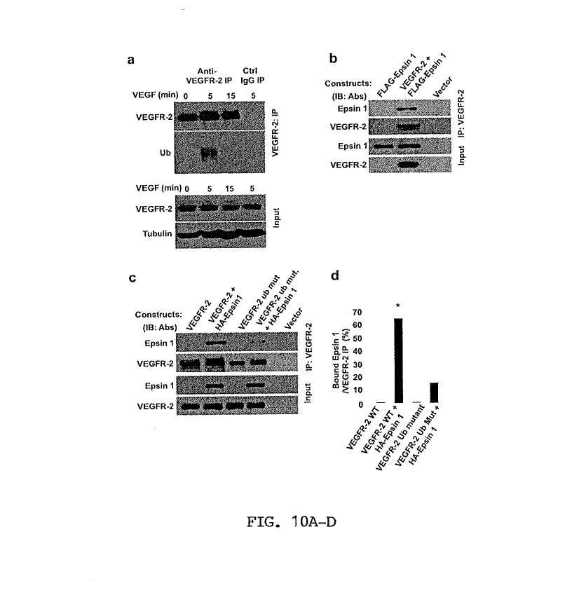

The inventors further hypothesize that epsins participate in the endocytosis of VEGFR-2 and directly regulate VEGF signaling switch off. To test this, they first investigate the molecular interaction between epsins and VEGFR-2 using co-immunoprecipitation (co-IP) experiments. VEGF stimulation induced VEGFR-2 ubiquitination (FIG. 10A) and epsin binding to VEGFR-2, with a maximum interaction occurring at 2 min after addition of VEGF in bovine aorta endothelial cells (BAEC) and binding to epsin 1 (FIGS. 3A-B) or epsin 2 (not shown), suggesting a redundant role of epsin 1 and 2. Co-IP from 293T cells co-expressing Flag-tagged epsin 1 and VEGFR-2 using either anti-Flag (FIG. 3C) or anti-VEGFR-2 (FIG. 10B) indicated that the epsin binds to activated VEGFR-2. Sequential immunoprecipitation experiments were employed to assess whether epsin binds ubiquitinated VEGFR-2. Cell lysates from 293T cells co-expressing Flag-tagged epsin 1 or HA-tagged epsin 1 and VEGFR-2 were extracted with RIPA buffer and immunoprecipitated using anti-Flag. The immunoprecipitates were then solubilized with SDS and after ‘renaturation’ by addition of excess RIPA buffer were processed for a second round of immunoprecipitation using anti-VEGFR-2 antibodies followed by western blot analysis with anti-ubiquitin antibodies. These experiments demonstrated that epsin 1 coprecipitated ubiquitinated VEGFR-2 (FIG. 3D). Conversely, a UIM-deficient mutant of epsin 1 failed to coprecipitate VEGFR-2 (FIG. 3E). Likewise, co-IP with a ubiquitination-deficient mutant of VEGFR-2 (FIG. 3F) with either anti-epsin (FIG. 3F) or anti-VEGFR-2 (FIGS. 10C-D) showed decreased binding of the ubiquitin-deficient mutant of VEGFR-2 to epsin 1, thus supporting UIM-ubiquitin interaction at the basis of the epsin/VEGFR-2 interaction. Since typically the fraction of a given surface protein that is in the ubiquitinated state is very low, it remains possible that interactions other than UIM-ubiquitin interactions, but triggered by such interactions, may account for the degree of coprecipitation observed. Consequently, confocal microscopy showed that 293T cells expressing the WT or ubiquitination-deficient mutant of VEGFR-2 exhibited impaired endocytosis of mutant but not WT VEGFR-2 induced by VEGF stimulation (FIG. 3G), suggesting that endocytosis of VEGFR-2 is correlated with its binding to epsins. Moreover, reconstitution of epsin 1 but not the UIM-deficient mutant of epsin 1 suppressed the enhanced phosphorylation of VEGFR-2 in DKO MECs (FIGS. 3H-I), suggesting that UIM-ubiquitin-mediated interaction of epsin and VEGFR-2 is critical for VEGFR-2 signaling switch off.

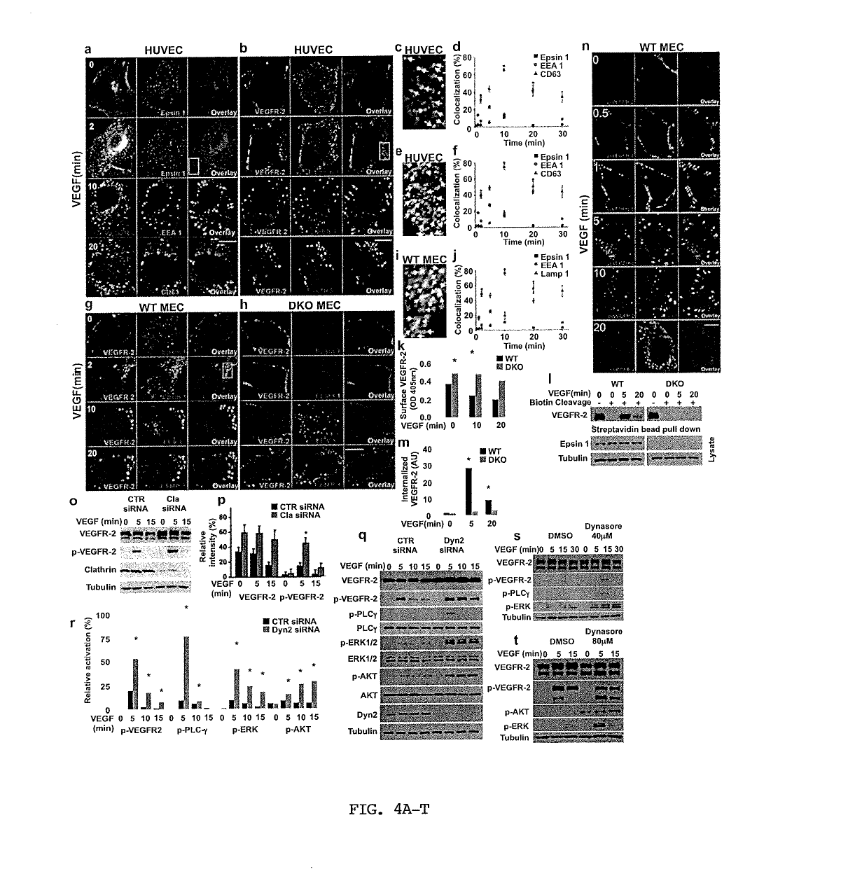

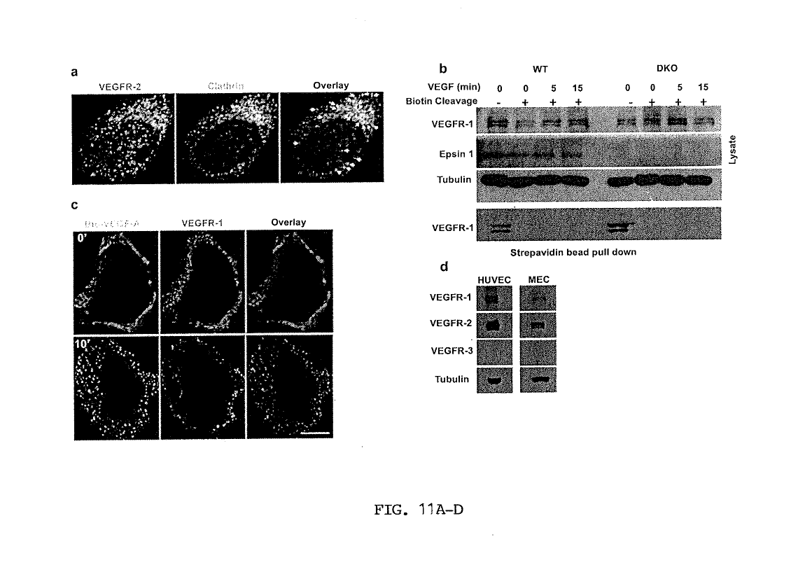

To directly test the role of epsin in the endocytosis and degradation of VEGFR-2, the inventors examined endocytic trafficking of VEGFR-2 upon VEGF-A stimulation in HUVEC by confocal microscopy. They observed maximal colocalization of VEGFR-2 with epsin or clathrin (FIG. 11A) at 2 min, an endosomal marker EEA1 at 10 min, and a lysosomal marker CD63 at 20 min stimulation (FIGS. 4A-F). Similar to HUVEC, VEGF-A induced VEGFR-2 endocytosis and colocalization with epsin, EEA1, and a lysosomal marker LAMP1 in WT MECs, but corresponding endocytic trafficking of VEGFR-2 was not observed in DKO MECs (FIGS. 4G-J). The impaired endocytosis of VEGFR-2 in DKO MECs was further demonstrated by biochemical assays using cell surface biotinylation.

VEGF treatment decreased surface VEGFR-2 in WT MECs but this action was very much reduced in DKO MECs (FIG. 4K). Accordingly, a prominent portion of endocytosed VEGFR-2 was detected in WT MECs at 5 min VEGF stimulation, however, this pool was decreased at 20 min, presumably due to lysosomal degradation (FIG. 4L). In contrast, very little internalized VEGFR-2 was detected in DKO MECs (FIG. 4L), indicating that epsins are key adaptor proteins in VEGFR-2 endocytosis and degradation. Interestingly, VEGF-A did not promote endocytosis of VEGFR-1, another VEGFR family member regardless of the presence and absence of epsins (FIGS. 11A-B), consistent with its negative regulatory role in angiogenesis as a secreted, catalytically inactive form. Additionally, VEGFR-3, which is highly expressed in lymphatic ECs, is at the limit of detection in either HUVEC or MECs (Jones et al., 2010) (FIG. 11C).

In contrast to a previous report (Sawamiphak et al., 2010), the inventors failed to observe that internalization is required for VEGFR-2 activation as the inventors detected activated VEGFR-2 at the cell surface that colocalized with total VEGFR-2 upon VEGF stimulation for either 0.5 or 1 min (FIG. 4N). Furthermore, similar to epsin inhibition, disruption of clathrin (FIGS. 4O-P) or dynamin 2 (FIGS. 4Q-R), two essential components in clathrin-mediated endocytosis (Schmid et al., 1998; Conner and Schmid, 2003; Slepnev and De Camilli, 2000), by siRNA-mediated knockdown or by an inhibitor to dynamin, Dynasore in HUVEC dramatically increased both total levels of VEGFR-2 and phosphorylation of VEGFR-2 and its downstream signaling effectors upon VEGF stimulation (FIGS. 4O-T), further supporting that ligand-engaged VEGFR-2 at the cell surface is fully active. In agreement with previous reports (Lampugnani et al., 2006; Lanahan et al., 2010), the inventors observed that endosomal but not lysosomal localized VEGFR-2 remains active (FIG. 4N), suggesting that internalized receptor tyrosine kinases (RTKs) might be delivered to endosomes where they form signaling complexes which may trigger qualitatively different signals than RTKs located at the plasma membrane (McPherson et al., 2001; von Zastrow and Sorkin, 2007; Seto et al., 2002; Di Fiore and De Camilli, 2001).

In vivo studies. In order to assess the in vivo efficacy of UIM treatment, several preclinical modeles were employed, including a melanoma model, a Lewis Lung carcinoma model, a prostate cancer model and a glioma brain tumor model.

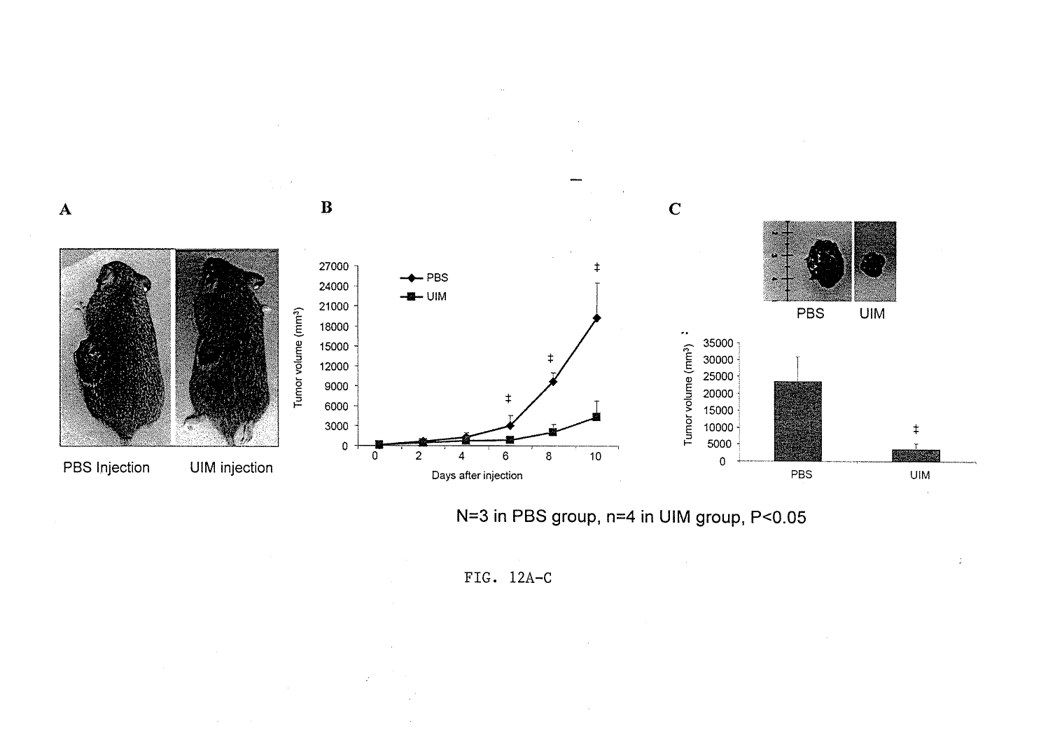

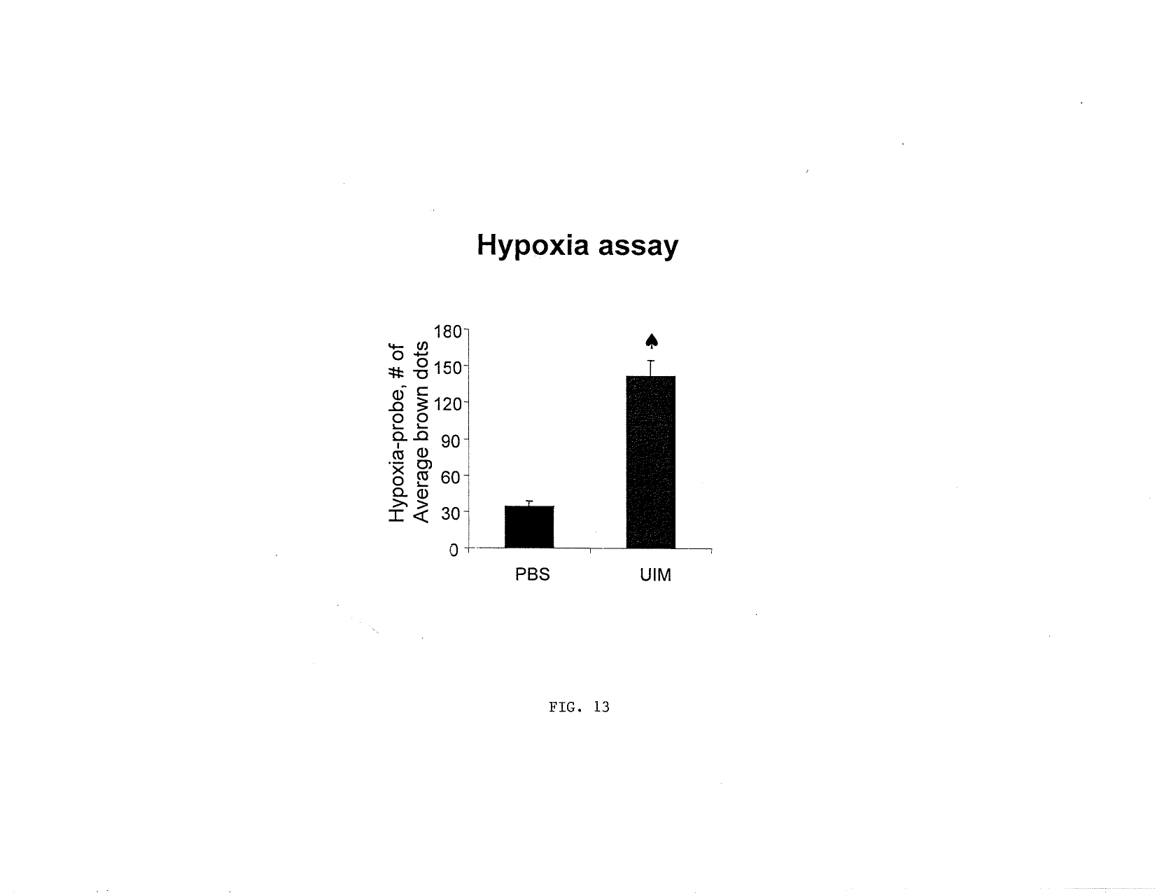

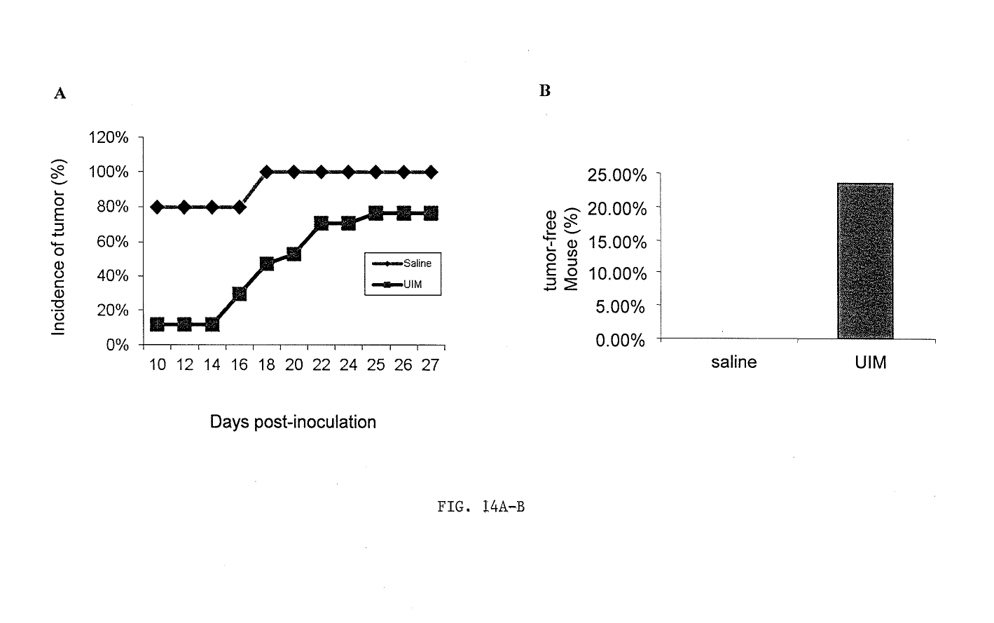

As shown in FIGS. 12A-C, melanoma tumor growth is significantly inhibited by 10-day consecutive injections of UIM peptide. Indeed, UIM treatment significantly increases tumor hypoxia and necrosis (FIG. 13), and UIM treatment significantly delays melanoma tumor incidence (FIGS. 14A-B). In addition, UIM treatment significantly increases non-productive angiogenesis revealed with blood vessel marker CD31 antibody staining, as well as produces dysfunctional angiogenesis revealed by CD31 staining and perfusion with FITC-Lectin and leaky vessels (data not shown).

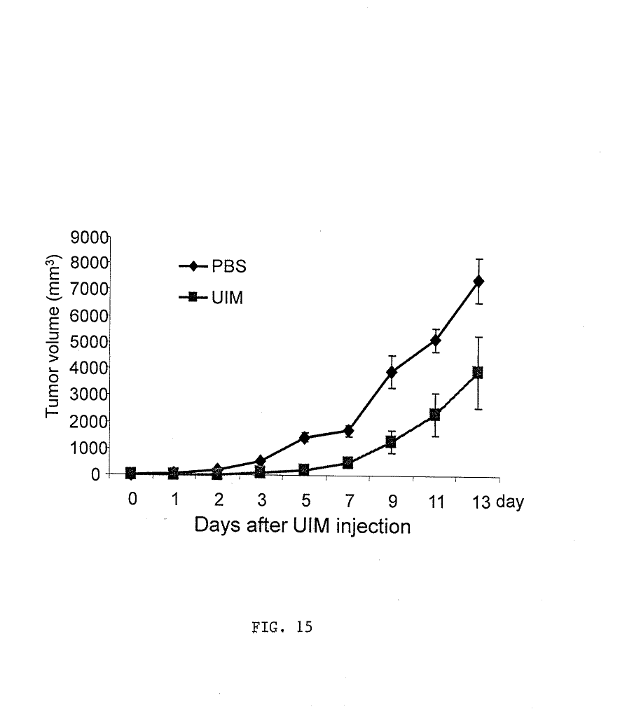

UIM treatment also inhibits LLC tumor growth (FIG. 15). CD31 staining is greatly increased by UIM treatment in LLC tumor, and hypoxia (red staining) is greatly increased by UIM treatment in LLC tumor, suggesting more necrosis in UIM treated LLC tumors (data not shown).

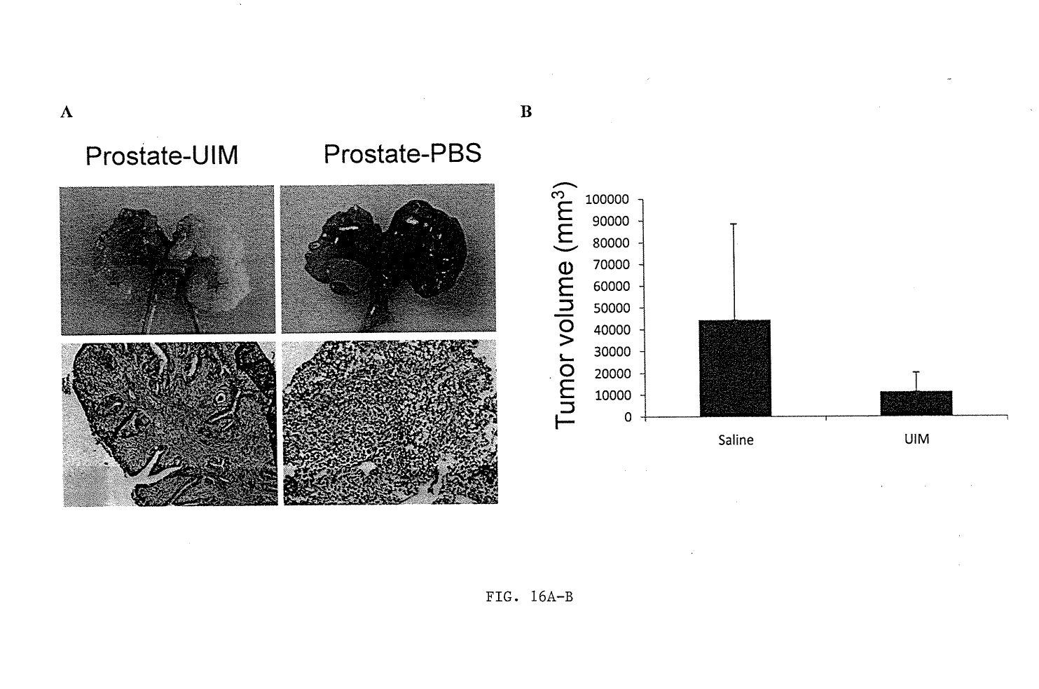

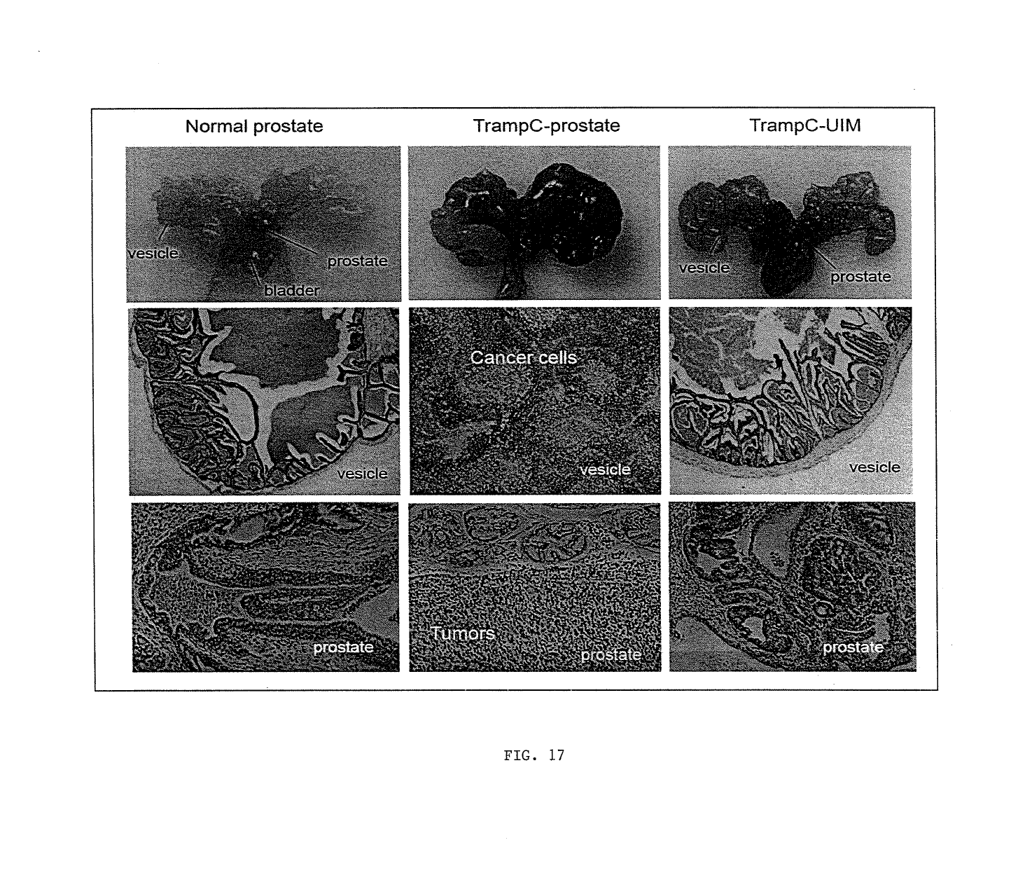

FIGS. 16A-B show that UIM treatment inhibits prostate tumor growth. Similarly, FIG. 17 shows that UIM peptide injection significantly inhibits tumor growth and improves prostate quality and surrounding vesicles.

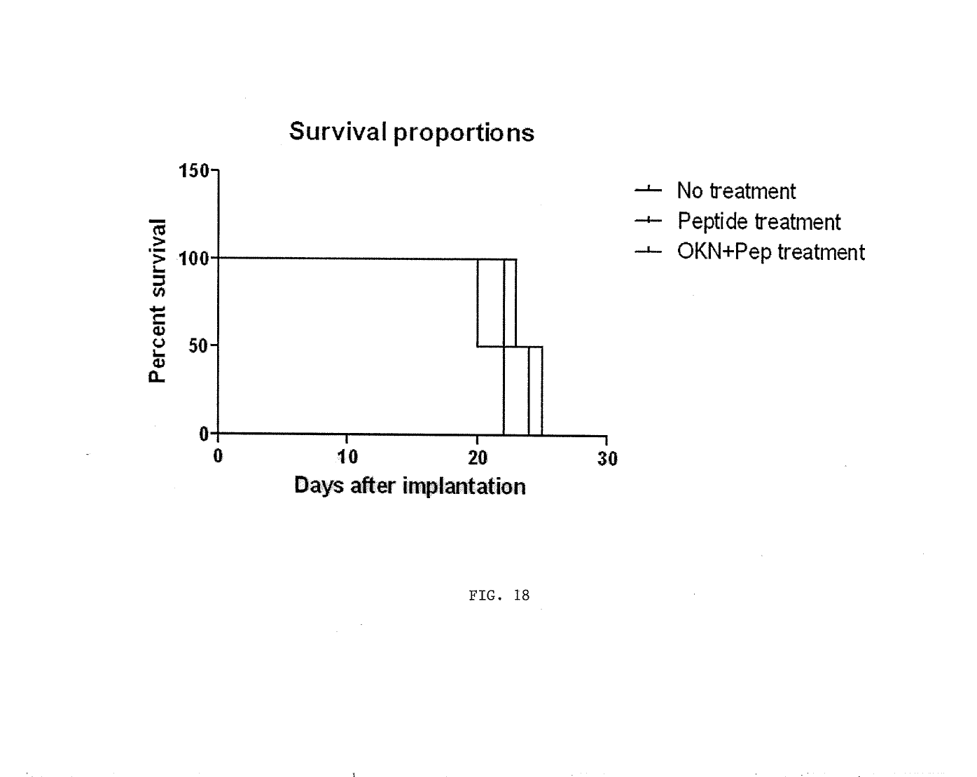

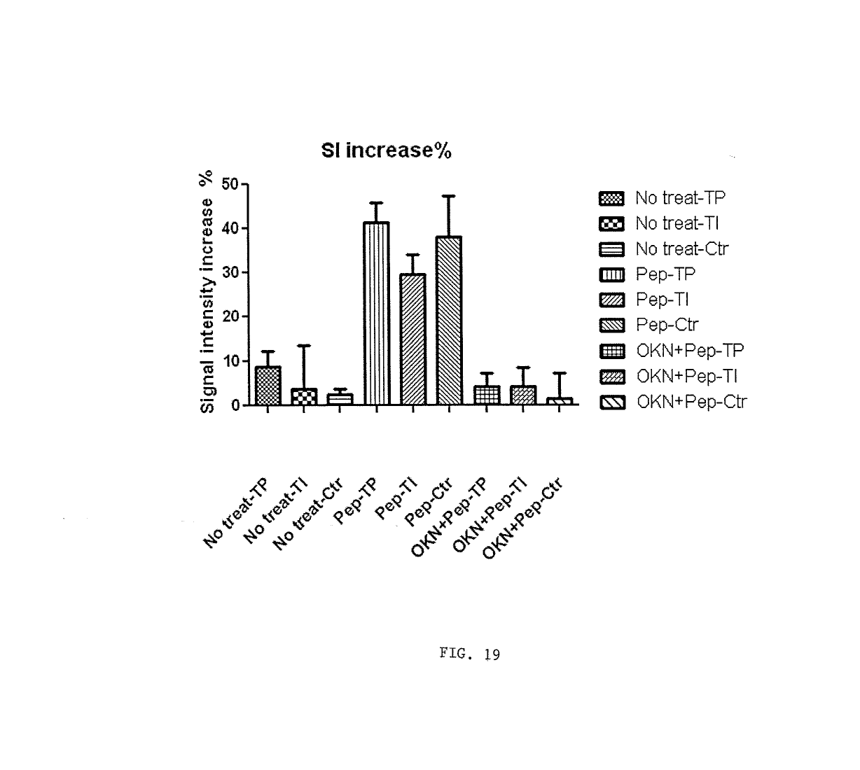

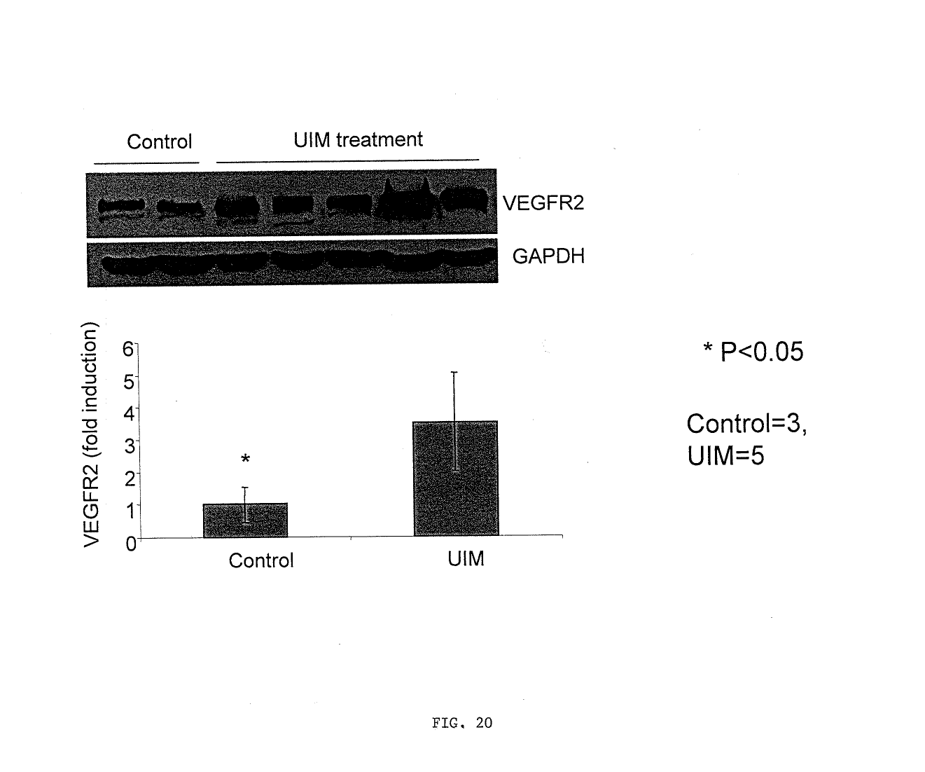

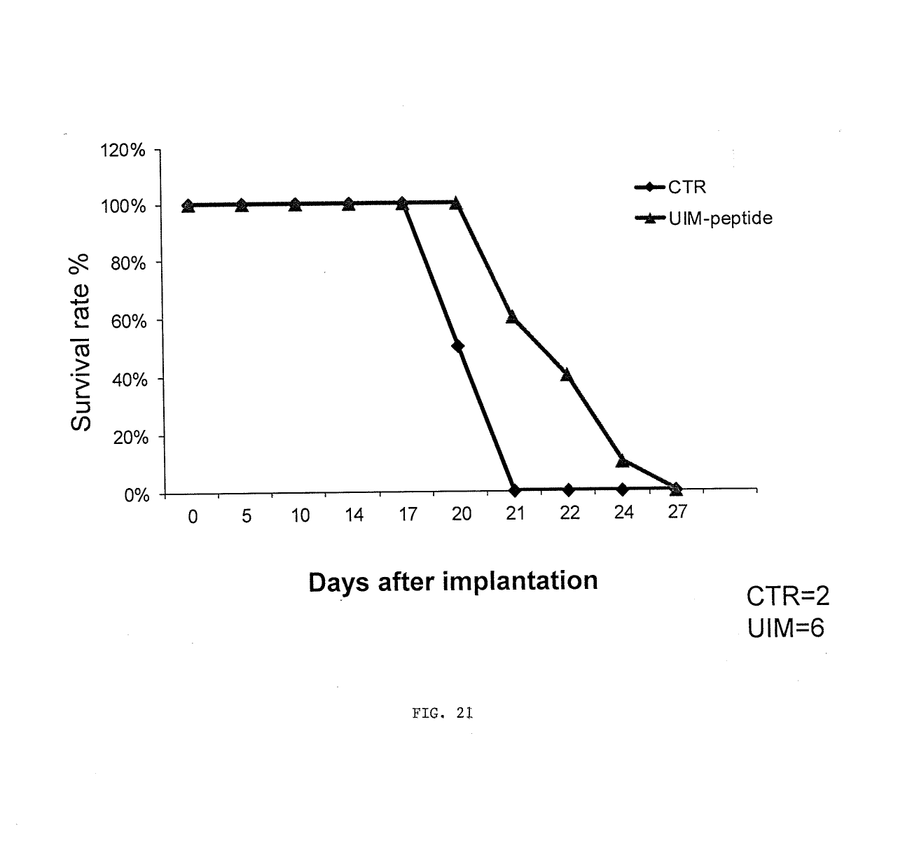

FIG. 18 shows UIM increases survival rate of mice bearing brain tumors. UIM treatment increases survival rate of mice bearing brain tumors. UIM treatment shows reduced brain tumor growth at day 17, and upregulates VEGFR2 level in brain tumor area revealed by molecular-targeting MRI using anti-VEGFR2 probe (data not shown). FIG. 19 shows that UIM upregulates VEGFR2 level in brain tumor area as revealed by functional and molecular-targeting MRI using anti-VEGFR2 probe, and OKN treatment cancels the effect of UIM on VEGFR2 levels. FIG. 20 shows that UIM treatment significantly increases VEGFR2 in GL6 brain tumors. UIM treatment produces greatly enlarged and disorganized non-productive vessels in GL6 brain tumor model (data not shown). FIG. 21 shows that UIM administration increases survival rate of mice bearing brain tumors.

Thus, the inventors observed significant tumor growth retardation and increased animal survival by UIM peptide treatment in melanoma, LLC, prostate cancer and glioma brain tumor preclinical models. Perturbation of tumor growth was mainly through the inhibitory action of UIM peptide on tumor angiogenesis by increasing VEGFR2 signaling producing hyper-dilated and hyper-leaky blood vessels, thereby prohibiting vessel perfusion, increasing hypoxia and tumor apoptosis.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### 7. REFERENCES

The following references, to the extent that they provide exemplary procedural or other details supplementary to those set forth herein, are specifically incorporated herein by reference.
U.S. Pat. No. 5,569,902
U.S. Pat. No. 5,488,145
U.S. Patent Publication 2007/0032453
Aguilar, Curr. Opin. Cellbiol., 15:184-190, 2003.
Barany and Merrifield, In: The Peptides, Gross and Meienhofer (Eds.), Academic Press, NY, 1-284, 1979.
Cao et al., Brain Res., 644:267-272, 1994.
Capaldi et al., Biochem. Biophys. Res. Comm., 74(2):425-433, 1977.
Carmeliet and Jain, Nature, 407(6801):249-257, 2000.
Carmeliet, Nat. Med., 9(6):653-660, 2003.
Carney et al., Proc. Natl. Acad. Sci. USA, 88:3633-3636, 1991.
Chen and De Camilli, Proc. Natl. Acad. Sci. USA, 102(8):2766-2771. 2005.
Chen and Zhuang, Proc. Natl. Acad. Sci. USA, 105(33):11790-11795, 2008.
Chen et al., Nature, 394(6695):793-797, 1998.
Chen et al., Proc. Natl. Acad. Sci. USA, 106(33):13838-13843, 2009.
Clough-Helfman et al., Free Radic. Res. Commun., 15:177-186, 1991.
Conner and Schmid, Nature, 422(6927):37-44, 2003.
Di Fiore and De Camilli, Cell, 106(1):1-4, 2001.
Duval et al., J. Biol. Chem., 278(22):20091-20097, 2003.
Ewan et al., Traffic, 7(9):1270-1282, 2006.
Floyd et al., Biochem. Biophys. Res. Commun., 74:79-84, 1977.
Floyd et al., FASEB J., 4:2587-2597, 1990.
Floyd et al., In: Neuroprotective Approaches to the Treatment of Parkinson's Disease and other Neurodegenerative Disorders, Chapman et al. (Eds.), Academic Press Limited, London, 69-90, 1996.
Floyd, Adv. Pharmacol., 38:361-378, 1997.
Folbergrova et al., Proc. Natl. Acad. Sci. USA, 92:5057-5061, 1995.
Ford et al., Nature, 419(6905):361-366, 2002.
Gerhardt et al., J. Cell Biol., 161(6):1163-1177, 2003.
Haglund et al., Nat. Cell Biol., 5(5):461-466, 2003.
Hawryluk et al., Traffic, 7(3):262-281, 2006.
Hellstrom et al., Cell Adh. Migr., 1(3):133-136, 2007.
Hellstrom et al., Nature, 445(7129):776-780, 2007.
Hensley et al., In: Neuroprotective Agents and Cerebral Ischaemia, Green and Cross (Eds.), Academic press Ltd., London, 299-317, 1996.
Hicke, Faseb. J., 11(14):1215-1226, 1997.
Hinton and Janzen, J. Org. Chem., 57:2646-2651, 1992.
Hofmann and Falquet, Trends Biochem. Sci., 26(6):347-350, 2001.
Itoh et al., Science, 291(5506):1047-1051, 2001.
Jain, Nat. Med., 9(6):685-693, 2003.
Jakobsson et al., Biochem. Soc. Trans., 37(Pt 6):1233-1236, 2009.
Janzen, Acc. Chem. Res., 4:31-40, 1971.
Jones et al., Arterioscler. Thromb. Vasc. Biol., 30(12):2553-2561, 2010.
Kazazic et al., Traffic, 10(2):235-245, 2009.
Kisanuki et al., Dev. Biol., 230(2):230-242, 2001.
Lampugnani et al., J. Cell Biol., 174(4):593-604, 2006.
Lanahan et al., Dev. Cell, 18(5):713-724, 2010.
McPherson et al., Traffic, 2(6):375-384, 2001.
Merrifield, Science, 232(4748):341-347 1986.
Monvoisin et al., Dev. Dyn., 235(12):3413-3422, 2006.
Murdaca et al., J. Biol. Chem., 279(25):26754-26761, 2004.
Noguera-Troise et al., Nature, 444(7122):1032-1037, 2006.
Novelli et al., In: Oxygen Free Radicals in Shock, Novelli and Ursini (Eds.), Karger, Basel, 119-124, 1986.
Olsson et al., Nat. Rev. Mol. Cell. Biol., 7(5):359-371, 2006.
Overstreet et al. Development, 131(21):5355-5366, 2004.
Pahlmark et al., Acta Physiol. Scand., 157:41-51, 1996.
Phng and Gerhardt, Dev. Cell, 16(2):196-208, 2009.
Phng et al., Dev. Cell, 16(1):70-82, 2009.
Pogrebniak et al., Surgery, 112:130-139, 1992.
Polo et al., Nature, 416(6879):451-455, 2002.
Poyer et al., 1978
Poyer et al., Biochim. Biophys. Acta, 539:402-409, 1978.
Ridgway et al., Nature, 444(7122):1083-1087, 2006.
Risau, Nature, 386(6626):671-674, 1997.
Rosenthal et al., J. Biol. Chem., 274(48):33959-33965, 1999.
Rossant and Howard, Annu. Rev. Cell Dev. Biol., 18:541-573, 2002.
Sawamiphak et al., Nature, 465(7297):487-491, 2010.
Schmid et al. Curr. Opin. Cell Biol., 10(4):504-512, 1998.
Seto et al., Genes Dev. 16(11):314-1336, 2002.
Shih et al., Nat. Cell Biol., 4(5):389-393, 2002.
Slepnev and De Camilli, Nat. Rev. Neurosci., 1(3):161-172, 2000.
Stewart and Young, In: i Solid Phase Peptide Synthesis, 2^{nd} Ed., Pierce Chemical Co., 1984.
Suchting et al., Proc. Natl. Acad. Sci. USA, 104(9):3225-3230, 2007.
Tam et al., J. Am. Chem. Soc., 105:6442, 1983.
Thurston and Kitajewski, Br. J. Cancer, 99(8):1204-1209, 2008.
Thurston et al., Nat. Rev. Cancer, 7(5):327-331, 2007.
Tian et al., Development, 131(23):5807-5815, 2004.
Traub, J. Cell Biol., 163(2):203-208, 2003.
von Zastrow and Sorkin, Curr. Opin. Cell Biol., 19(4):436-445, 2007.
Wang and Struhl, Development, 131(21):5367-5380, 2004.
Wang and Struhl, Development, 132(12):2883-2894, 2005.
Weinstein and Lawson, Cold Spring Harb. Symp. Quant. Biol., 67:155-162, 2002.
Wendland et al., Embo. J., 18(16):4383-4393, 1999.
Wendland, Nature Rev. Molec. Cell Biol., 3:971-977, 2002.
Williams et al., Blood, 107(3):931-939, 2006.
Zhang et al., J. Clin. Invest., 118(12):3904-3916, 2008.

What is claimed is:

1. A method of treating cancer in a subject comprising administering to said subject an ubiquitin interactive motif (UIM)-containing peptide.

2. The method of claim 1, wherein administration is intra-tumoral, regional to a tumor, or systemic.

3. The method of claim 2, wherein systemic administration is oral, intravenous, or intaarterial.

4. The method of claim 1, wherein said cancer is recurrent, metastatic or multidrug resistant.

5. The method of claim 1, wherein the cancer is brain cancer, head & neck cancer, throat cancer, nasopharyngeal cancer, esophageal cancer, lung cancer, stomach cancer, liver cancer, pancreatic cancer, colon cancer, rectal cancer, prostate cancer, testicular cancer, ovarian cancer, uterine cancer, cervical cancer, breast cancer, or skin cancer.

6. The method of claim 1, wherein treating comprises reducing tumor growth, reducing tumor size, reducing tumor burden, inducing apoptosis in cancer cells, inhibiting tumor tissue invasion, or inhibiting metastasis.

7. The method of claim 1, wherein said UIM-containing peptide comprises the sequence X—Ac-Ac—Ac-Ac-Hy-X—X-Ala-X—X—X-Ser-X—X—Ac—X—X—X—X, where Hy represents a large hydrophobic residue (typically Leu), Ac represents an acidic residue (Glu, Asp), and X represents residues that are less well conserved.

8. The method of claim 1, further comprising a secondary anti-cancer therapy.

9. The method of claim 8, wherein the secondary anti-cancer therapy is radiation, surgery, chemotherapy, hormone therapy, immunotherapy, or toxin therapy.

10. The method of claim 8, wherein the secondary anti-cancer therapy is 2,4-disulfonyl phenyl tert-butyl nitrone (2,4-ds-PBN).

11. A method of inducing non-productive vessel formation in a subject comprising administering to said subject an ubiquitin interactive motif (UIM)-containing peptide.

12. The method of claim 11, wherein administration is oral, intramuscular, subcutaneous, intravenous, or intaarterial.

13. The method of claim 11, wherein said subject has cancer.

14. The method of claim 13, wherein said cancer is recurrent, metastatic or multidrug resistant.

15. The method of claim 13, wherein the cancer is brain cancer, head & neck cancer, throat cancer, nasopharyngeal cancer, esophageal cancer, lung cancer, stomach cancer, liver cancer, pancreatic cancer, colon cancer, rectal cancer, prostate cancer, testicular cancer, ovarian cancer, uterine cancer, cervical cancer, breast cancer, skin cancer or a blood cancer.

16. The method of claim 11, wherein the subject has a non-cancer neovascular disease.

17. The method of claim 16, wherein said non-cancer neovascular disease is retinal neovascularization, haemorrhagic telangiectasia (HHT), neurofibromatosis type 1, familial cavernous malformation, and forms of lymphangiogenesis.

18. The method of claim 11, wherein said UIM-containing peptide comprises the sequence X—Ac-Ac—Ac-Ac-Hy-X—X-Ala-X—X—X-Ser-X—X—Ac—X—X—X—X, where Hy represents a large hydrophobic residue (typically Leu), Ac represents an acidic residue (Glu, Asp), and X represents residues that are less well conserved.

19. The method of claim 17, further comprising a secondary treatment.

20. The method of claim 19, wherein the secondary treatment is ruboxistaurine, VEGI IL-20, ranibizumab, bevacizumab or pegaptanib.

21. A pharmaceutical composition comprising a ubiquitin interactive motif (UIM)-containing peptide dispersed in a pharmalogically acceptable medium, carrier or diluent.

22. The composition of claim 21, wherein said peptide comprises the sequence X—Ac-Ac—Ac-Ac-Hy-X—X-Ala-X—X—X-Ser-X—X—Ac—X—X—X—X, where Hy represents a large hydrophobic residue (typically Leu), Ac represents an acidic residue (Glu, Asp), and X represents residues that are less well conserved.

23. The composition of claim 21, wherein said peptide is about 20-30 residues in length.

24. The composition of claim 21, wherein said peptide is 21, 22, 23, 24, 25, 26, 27, 28, 29 or 30 residues in length.

25. The composition of claim 21, formulated in a lipid carrier.


SEQUENCE LISTING (15 sequences)

SEQ ID NO: 1 - RNA, 25 nt, Artificial Sequence
  Synthetic primer
ugcucuucucggcucaaacuaaggg

SEQ ID NO: 2 - RNA, 25 nt, Artificial Sequence
  Synthetic primer
aaauccaacagcguagucugcugug

SEQ ID NO: 3 - RNA, 25 nt, Artificial Sequence
  Synthetic primer
cgcgguuacuugagaugaaccuuau

SEQ ID NO: 4 - RNA, 19 nt, Artificial Sequence
  Synthetic primer
ggauauugagggcaagaag

SEQ ID NO: 5 - RNA, 19 nt, Artificial Sequence
  Synthetic primer
gcgaaucgucaccacuuac

SEQ ID NO: 6 - DNA, 20 bp, Artificial Sequence
  Synthetic primer
acaccggacaaaccaaagac

SEQ ID NO: 7 - DNA, 20 bp, Artificial Sequence
  Synthetic primer
gtcacctcgttcatgcactc

SEQ ID NO: 8 - DNA, 17 bp, Artificial Sequence
  Synthetic primer
catgaagagagctcacc

SEQ ID NO: 9 - DNA, 17 bp, Artificial Sequence
  Synthetic primer
aatgtgtccgaggccac

SEQ ID NO: 10 - DNA, 24 bp, Artificial Sequence
  Synthetic primer
gacaactacctctcagattatggc

SEQ ID NO: 11 - DNA, 18 bp, Artificial Sequence
  Synthetic primer
cgggagcatgggaaaagc

SEQ ID NO: 12 - DNA, 20 bp, Artificial Sequence
  Synthetic primer
ggtccccactgcctttgaga

SEQ ID NO: 13 - DNA, 21 bp, Artificial Sequence
  Synthetic primer
aggatggcgagctgactgttc

SEQ ID NO: 14 - DNA, 20 bp, Artificial Sequence
  Synthetic primer
gacggccaggtcatcactat

SEQ ID NO: 15 - DNA, 20 bp, Artificial Sequence
  Synthetic primer
acatctgctggaaggtggac